(19) United States
(12) Patent Application Publication
(10) Pub. No.: US 2013/0304108 A1
(43) Pub. Date: Nov. 14, 2013

(54) SYSTEMS AND APPARATUS FOR TREATING BLOOD VESSELS AND RELATED METHODS

(71) Applicants: Daniel C. Weber, Plymouth, MN (US); Chad J. Kugler, Buffalo, MN (US); Allen W. Groenke, Bloomington, MN (US)

(72) Inventors: Daniel C. Weber, Plymouth, MN (US); Chad J. Kugler, Buffalo, MN (US); Allen W. Groenke, Bloomington, MN (US)

(21) Appl. No.: 13/466,853

(22) Filed: May 8, 2012

(51) Int. Cl. A61M 29/00

(52) U.S. Cl. 606/198

(57) ABSTRACT

The present disclosure is directed to a system for treating a blood vessel including a blood vessel lumen defined by a blood vessel wall, the blood vessel lumen being at least partially obstructed. The system may include a shaft assembly including an orienting element. The system may also include a re-entry device extending into the central lumen. The re-entry device may comprise a core wire configured such that bending stresses created in the core wire during bending about a design bend radius are less than the elastic limit of the core wire so that the core wire will elastically recover from the bending upon release.

### FIELD OF THE INVENTION

This disclosure relates to systems and devices for treating chronic occlusions in blood vessels and associated methods. More particularly, the disclosure relates to devices for establishing a blood flow path around a chronic total occlusion and methods for fabricating those devices.

### BACKGROUND OF THE INVENTION

A number of diseases are caused by the build-up plaque in the arteries. These plaque deposits limit blood flow to the tissues that are supplied by that particular artery. When these deposits build up in the arteries of the heart, the problem is called coronary artery disease (CAD). When these deposits build up in the arteries of a limb, such as a leg, the condition is called peripheral artery disease (PAD).

Peripheral artery disease affects 8 to 12 million individuals in the United States and is also prevalent in Europe and Asia. Roughly 30% of the population over the age of 70 suffers from PAD. PAD typically causes muscle fatigue or pain brought about by exertion and relieved by rest. Symptoms of PAD can include leg pain during walking and wounds that do not heal. The inability to walk without leg pain often causes patients to stop exercising and reduces the patient's mobility. When the plaque builds up to the point where an artery is totally occluded, the obstruction is referred to as a Chronic Total Occlusion (CTO). A CTO that occludes the peripheral arteries for PAD patients is extremely serious. PAD patients that suffer from a CTO often enter a downward spiral towards death. Often the CTO in a peripheral artery results in limb gangrene, which can require limb amputation to resolve. The limb amputation in turn causes other complications, and roughly half of all PAD patients die within two years of a limb amputation.

The blood pumping action of the heart muscle is critical to sustaining the life of a patient. In order for the heart to function properly, the tissues of the heart muscle must be continuously supplied and re-supplied with oxygen. To receive an adequate supply of oxygen, the heart muscle must be well perfused with blood. In a healthy heart, blood perfusion is accomplished with a system of arteries and capillaries. However, due to age, high cholesterol and other contributing factors, a large percentage of the population has arterial atherosclerosis that totally occludes portions of the patient's coronary arteries. A chronic total occlusion (CTO) in a coronary artery may cause painful angina, atrophy of cardiac tissue and patient death.

### SUMMARY

The present disclosure is directed to a system for treating a blood vessel including a blood vessel lumen defined by a blood vessel wall, the blood vessel lumen being at least partially obstructed. The system may include a shaft assembly including an orienting element. The orienting element may have an expanded shape dimensioned such that, when the orienting element assumes the expanded shape within the blood vessel wall, the shaft assembly will assume an arbitrary one of two possible orientations relative to the blood vessel lumen. The two possible orientations may comprise a first orientation and a second orientation. The shaft assembly may define a shaft lumen and a first aperture and a second aperture. The first aperture may be positioned to face the blood vessel lumen when the shaft assembly assumes the first orientation, and the second aperture may be positioned to face the blood vessel lumen when the shaft assembly assumes the second orientation. The system may also include a re-entry device extending into the central lumen. The re-entry device may comprise a core wire configured such that bending stresses created in the core wire during bending about a design bend radius are less than the elastic limit of the core wire so that the core wire will elastically recover from the bending upon release.

The disclosure is also directed to a method for treating a blood vessel including a blood vessel lumen defined by a blood vessel wall, the blood vessel lumen being at least partially obstructed. The method may include creating a strengthened region in a wire and assembling a re-entry device including the wire. The re-entry device may have a distal end. The method may also include instructing a user of the re-entry device to insert the distal end into a lumen defined by an orienting catheter that is extending along the blood vessel, position the distal end proximate a first aperture, and rotate the re-entry device until the distal end enters the first aperture. The strengthened region of the wire may be configured such that bending stresses created in the wire during bending about a design bend radius are less than the elastic limit of the wire so that the wire will elastically recover from the bending upon withdrawal from the lumen of the orienting catheter.

### BRIEF DESCRIPTION OF THE DRAWINGS

FIG. 1 is a stylized anterior view showing a human patient. A portion of the patient's arterial system is schematically illustrated in FIG. 1.

FIG. 2A is an enlarged schematic view showing a portion of the arterial system of a patient who has been treated for peripheral artery disease (PAD). FIG. 2B is an enlarged schematic view showing a portion of the arterial system of a patient who has been treated for coronary artery disease (CAD).

FIG. 3A is a perspective view showing a system that may be used, for example, to establish a blood flow path between a proximal segment of a blood vessel and a distal segment of a blood vessel that are separated by a chronic total occlusion. The system of FIG. 3A includes a re-entry device and an orienting device. FIG. 3B is an enlarged isometric view further illustrating a portion of the system shown in FIG. 3A. FIG. 3C is a cross-sectional view illustrating a lateral cross-section of the system shown in FIG. 3A.

FIG. 4A and FIG. 4B are stylized cross-sectional views schematically illustrating the structure of a wire. In the embodiment of FIG. 4A, no cold working has been performed on the wire. In the embodiment of FIG. 4B, the wire has been subjected to a cold working process. FIG. 4C is an exemplary stress-strain diagram. FIG. 4D is a stress-strain diagram illustrating changes occurring in the mechanical behavior of the material in a strengthened region of a wire that has been processed in accordance with this detailed description.

FIG. 5 is cross-sectional view showing a re-entry device in accordance with the detailed description.

FIG. 6 is an enlarged plan view further illustrating the re-entry device shown in the previous figure.

FIG. 7 is a plan view of an exemplary re-entry device in accordance with the detailed description.

FIG. 8 is a cross-sectional view of an exemplary re-entry device in accordance with the detailed description.

FIG. 9 is a cross-sectional view of an exemplary re-entry device in accordance with the detailed description.

FIG. 10 through FIG. 20 are a series of stylized fragment views illustrating various steps that may be included as part of the methods in accordance with the detailed description.

FIG. 21A, FIG. 21B, and FIG. 21C are stylized plan views illustrating an exemplary process that may be used to form a strengthened region in a wire 160. FIG. 21B is taken from a viewpoint that is generally orthogonal the viewpoint used to create FIG. 21A. FIG. 21C is created from the viewpoint illustrated by a line C-C in FIG. 21A.

FIG. 22 is a stylized perspective view showing a wire having an outer surface. A plurality of spots can be seen on the outer surface of the wire. The spots shown in FIG. 22 form a pattern of overlapping spots that substantially covers the outer surface of the wire.

FIG. 23 is a plan view illustrating a process that may be used to form a strengthened region in a wire.

FIG. 24A is a stylized perspective view illustrating a first series of overlapping laser beam spots that form a first generally helical path around a wire. FIG. 24B is a stylized perspective view illustrating a second series of overlapping laser beam spots that form a second generally helical path around the wire. The spots of the first series and the spots of the second series may be combined to form a pattern of overlapping spots that substantially covers the outer surface of wire.

FIG. 25A is a stylized perspective view of a wire section. FIG. 25B is a second stylized perspective view of the wire section 180 shown in FIG. 25A. A first torque and a second torque are placing the wire section in torsion in the embodiment of FIG. 25B.

FIG. 26 is cross-sectional view showing an exemplary re-entry device in accordance with the detailed description.

FIG. 27 is cross-sectional view showing another exemplary a re-entry device.

### DETAILED DESCRIPTION

The following detailed description should be read with reference to the drawings in which similar elements in different drawings are numbered the same. The drawings depict illustrative embodiments and are not intended to limit the scope of the invention.

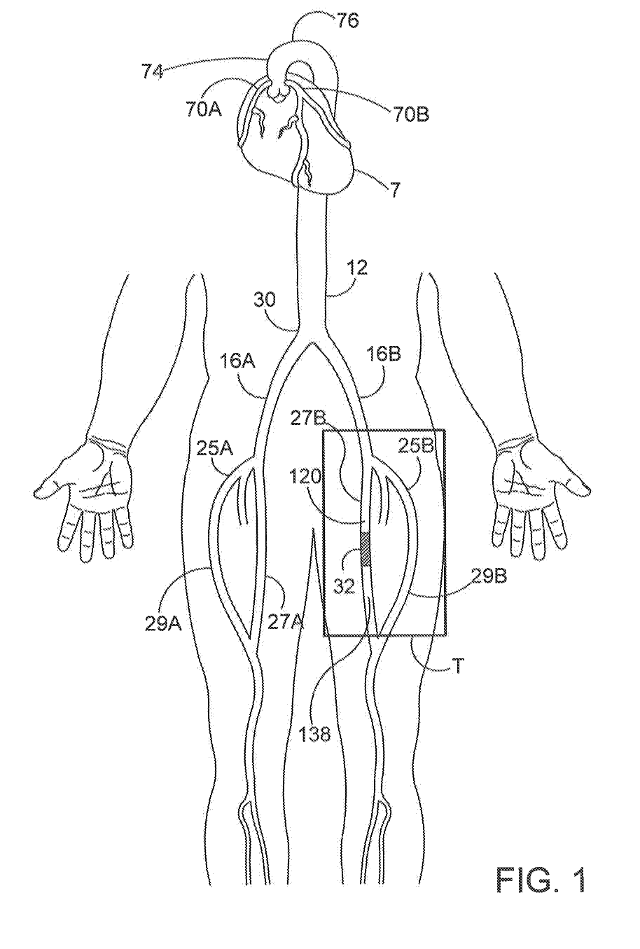

FIG. 1 is a stylized anterior view illustrating the cardiovascular system of a human patient. The cardiovascular system of FIG. 1 includes a heart 7 that pumps blood and an arterial system that distributes oxygen rich blood throughout the body. During each heartbeat, the left ventricle of heart 7 contracts, pumping blood through the aortic valve and into the ascending aorta 74. Blood from the ascending aorta 74 flows through the aortic arch 76 and down the descending aorta 12 to the lower body. Blood from the ascending aorta 74 also flows into the left coronary artery 70B and the right coronary artery 70A. In a healthy heart, the left coronary artery 70B and the right coronary artery 70A provide a continuous flow of blood into the heart which assures that the heart muscle remains well oxygenated.

The descending aorta 12 gives off numerous branches that supply oxygenated blood to the chest cage and the organs within the chest. The descending aorta 12 continues to the iliac bifurcation 30, which is a branch that splits into the two common iliac arteries 16A and 16B. The iliac arterial vasculature includes two branches continuing from the iliac bifurcation 30. The right branch includes the right common iliac artery 16A, which bifurcates into the right external iliac artery 25A and the right internal iliac artery 27A. When the right external iliac artery 25A passes posterior to the inguinal ligament, it becomes the right femoral artery 29A of the right leg. The left branch of the iliac arterial vasculature includes the left common iliac artery 16B, which bifurcates into the left external iliac artery 25B and the left internal iliac artery 27B. When the left external iliac artery 25B passes posterior to the inguinal ligament, it becomes the left femoral artery 29B of the left leg.

In the exemplary embodiment of FIG. 1, an occlusion 32 is blocking blood flow through a portion of a blood vessel within a target region T of the patient's arterial system. The occlusion 32 is obstructing blood flow between a proximal segment 120 of the blood vessel and a distal segment 138 of the blood vessel. A therapy system in accordance with the present detailed description may be used to establish a blood flow path between proximal segment 120 and distal segment 138.

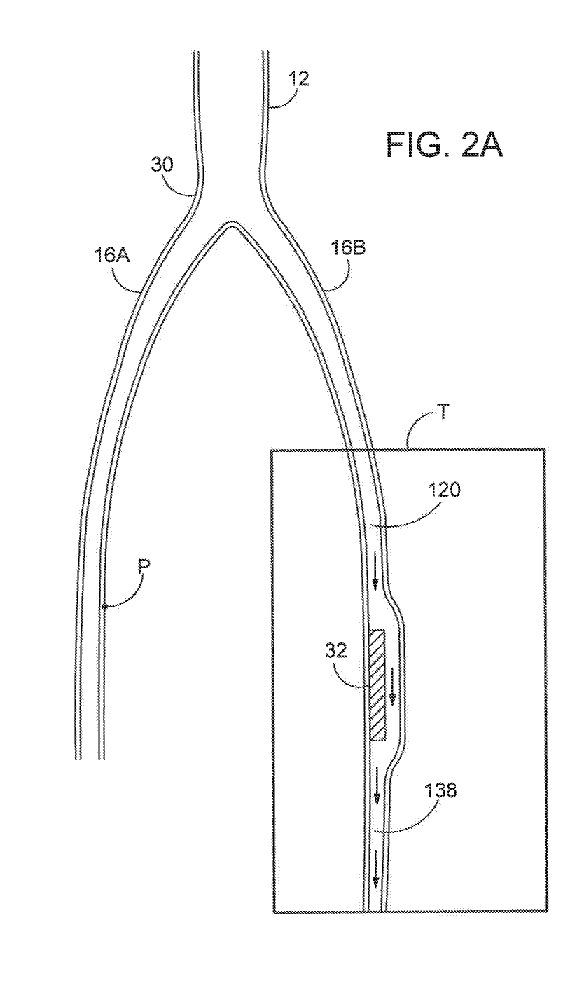

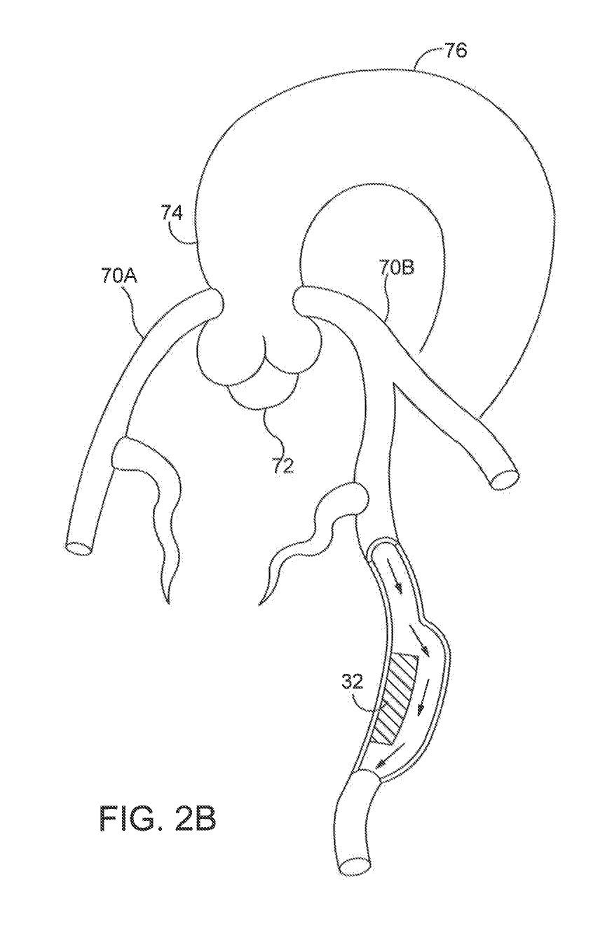

FIG. 2A is an enlarged schematic view showing a portion of the arterial system of a patient who has been treated for peripheral artery disease (PAD). The portion of the arterial system shown in FIG. 2A includes the descending aorta 12, the iliac bifurcation 30, the right common iliac artery 16A and the left common iliac artery 16B. In the exemplary embodiment of FIG. 2A, the patient's condition has been treated by establishing a blood flow path around an occlusion 32. The blood flow around occlusion 32 is illustrated using arrows in FIG. 2A. The portion of the arterial system located in target region T may be treated using a transradial approach. When using the transradial approach, an endovascular device may enter the vascular system at an access point P. After entering the arterial system, the endovascular device may be advanced through iliac bifurcation 30 to reach the target region T in the leg opposite the leg that is the site of access.

FIG. 2B is an enlarged schematic view showing a portion of the arterial system of a patient who has been treated for coronary artery disease (CAD). The portion of the arterial system shown in FIG. 2B includes the aortic valve 72, the right coronary artery 70A, the left coronary artery 70B, the ascending aorta 74, and the aortic arch 76. Left coronary artery 70B and right coronary artery 70A each meet the ascending aorta 74 at an ostium. During the systolic phase of each cardiac cycle, oxygen rich blood from the ascending aorta 74 flows through left coronary artery 70B and right coronary artery 70A. In a healthy heart, this oxygen rich blood is distributed throughout the heart by a network of arteries and capillaries.

In the exemplary embodiment of FIG. 2B, the patient's condition has been treated by establishing a blood flow path around an occlusion 32. The blood flow around occlusion 32 is illustrated using arrows in FIG. 2B. In the exemplary embodiment of FIG. 2B, occlusion 32 is located in left coronary artery 70B. The methodology for treating a coronary artery may include inserting a guide catheter into a femoral artery and advancing the guide catheter such that its distal tip moves through that artery, up the descending aorta, through the aortic arch and ultimately into the ostium of the coronary artery. A therapy system in accordance with this detailed description may then be advanced through the guide catheter into the coronary artery. Once in the coronary artery, the system may be used to establish a blood flow path between a proximal segment of the coronary artery and distal segment of the coronary artery.

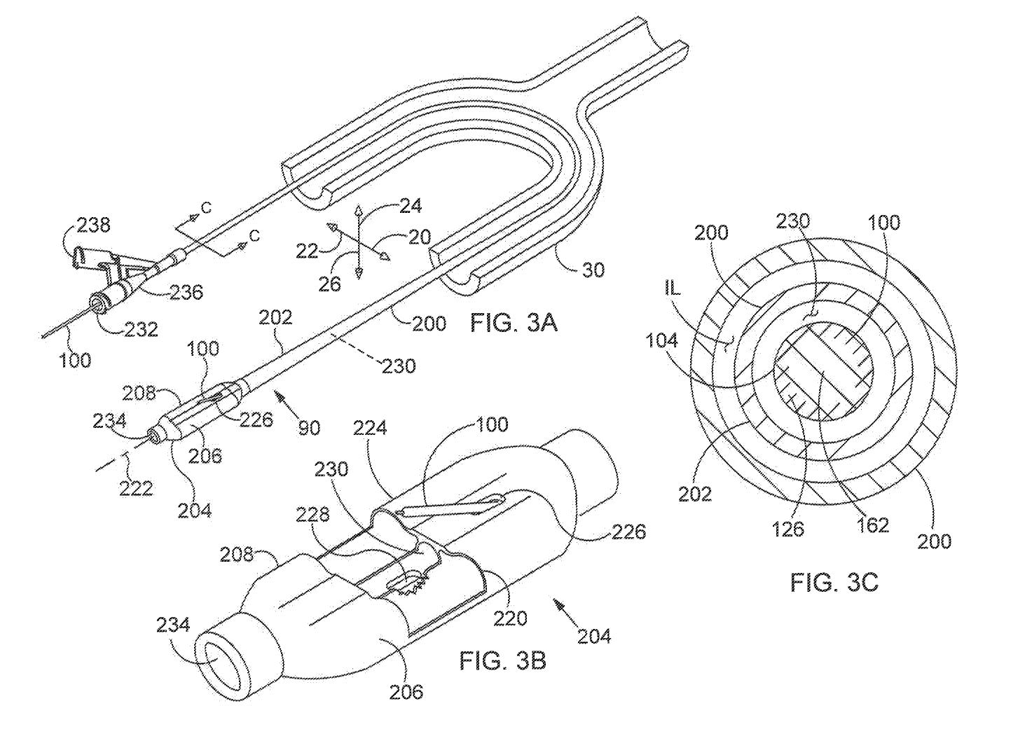

FIG. 3A is a perspective view showing a therapy system 90 including an orienting catheter 200 and a re-entry device 100. Therapy system 90 may be used, for example, to establish a blood flow path between a proximal segment of a blood vessel and a distal segment of a blood vessel that are separated by a chronic total occlusion. FIG. 3B is an enlarged isometric view further illustrating a portion of therapy system 90. FIG. 3C is an enlarged cross-sectional view illustrating a lateral cross-section of therapy system 90. The lateral cross-section shown in FIG. 3C is created by cutting therapy system 90 along section line C-C shown in FIG. 3A. FIG. 3A, FIG. 3B, and FIG. 3C may be collectively referred to as FIG. 3.

Orienting catheter 200 of FIG. 3 comprises a shaft assembly 202 and an orienting element 204 that is carried by shaft assembly 202. Orienting element 204 is capable of assuming both a collapsed shape and an expanded shape. Orienting element 204 may be selectively placed in the collapsed shape, for example, while the orienting element is being advanced past an occlusion. Orienting element 204 may be selectively placed in the expanded shape, for example, while the orienting catheter 200 is being used to direct re-entry device 100 toward the lumen of a blood vessel. In FIG. 3, orienting element 204 is shown assuming the expanded shape.

Orienting element 204 of orienting catheter 200 comprises a first portion 206 and a second portion 208. In the embodiment of FIG. 3, first portion 206 of orienting element 204 comprises a first inflatable member 220. Second portion 208 of orienting element 204 comprises a second inflatable member 224 in the embodiment of FIG. 3.

First inflatable member 220 of orienting element 204 extends in a first direction 20 away from longitudinal axis 222 of shaft assembly 202. Second inflatable member 224 of orienting element 204 extends away from longitudinal axis 222 of shaft assembly 202 in a second direction 22. First direction 20 and second direction 22 are represented with arrows in FIG. 3. With reference to FIG. 3, it will be appreciated that second direction 22 is generally opposite first direction 20. In FIG. 3, the arrows representing first direction 20 and second direction 22 are directed about 180 degrees away from one another.

Shaft assembly 202 of FIG. 3 defines a first aperture 226 and a second aperture 228. In the embodiment of FIG. 3, first aperture 226 extends away from central lumen 230 in a third direction 24. Second aperture 228 extends away from central lumen 230 in a fourth direction 26. Third direction 24 and fourth direction 26 are represented with arrows in FIG. 3. In the embodiment of FIG. 3, third direction 24 and fourth direction 26 extend in generally opposite directions. In FIG. 3, the arrows representing third direction 24 and fourth direction 26 are directed about 180 degrees away from each other. It is contemplated that third direction 24 and fourth direction 26 are generally orthogonal to first direction 20 and second direction 22.

A hub 236 is fixed to the proximal end of shaft assembly 202. Hub 236 includes an inflation port 238. Inflation port 238 fluidly communicates with an interior of first inflatable member 220 and second inflatable member 224 via inflation lumens IL defined by shaft assembly 202. The inflatable members may be inflated by injecting an inflation media into inflation port 238. Examples of inflation media that may be suitable in some applications include saline, carbon dioxide, and nitrogen.

Orienting catheter 200 defines a proximal port 232, a distal port 234 and a central lumen 230 that extends between proximal port 232 and distal port 234. In the embodiment of FIG. 3, proximal port 232 is defined by hub 236 and distal port 234 is defined by shaft assembly 202. Re-entry device 100 may be inserted into proximal port 232, advanced along central lumen 230, and advanced through any one of distal port 234, first aperture 226 and second aperture 228.

A lateral cross-section of therapy system 90 is shown in FIG. 3C. With reference to FIG. 3C, it will be appreciated that re-entry device 100 comprises a core wire 104 that is disposed in a central lumen 230 defined by shaft assembly 202 of orienting catheter 200. Core wire 104 of re-entry device 100 comprises a strengthened region 126 that is illustrated using crosshatched lines in FIG. 3C. In FIG. 3C, strengthened region 126 encircles a central region 162 of core wire 104.

The material in strengthened region 126 has a first elastic limit and the material in central region 162 has a second elastic limit. In some useful embodiments, the first elastic limit is greater than the second elastic limit. When this is the case, the material in the strengthened region 126 has greater resistance to plastic deformation produced by bending stresses created when the wire is extending through a bend (e.g., the iliac bifurcation) in the vasculature of a patient. The material in strengthened region 126 has a first level of ductility, and the material in central region 162 has a second level of ductility. In some useful embodiments, the second level of ductility is greater than the first level of ductility. A central portion having a relatively high level of ductility may provide the wire with a desirable level of toughness.

In FIG. 3, orienting catheter 200 and re-entry device 100 can be seen extending through an iliac bifurcation 30. As described previously, iliac bifurcation 30 is part of the vasculature of a patient. With reference to FIG. 3, it will be appreciated that orienting catheter 200 and re-entry device 100 must bend as they extend through iliac bifurcation 30. The relatively high elastic limit in the strengthened region 126 of core wire 104 makes it less likely that core wire 104 will experience plastic deformation as bending forces are applied to core wire 104. The material in strengthened region 126 has a first elastic modulus, and the material in central region 162 has a second elastic modulus. In some useful embodiments, the first elastic modulus is greater than the second elastic modulus.

A therapy system in accordance with the present detailed description may be used to establish a blood flow path around an occlusion in a blood vessel. During a procedure, the physician may selectively insert the distal end of the re-entry device into the first aperture and/or the second aperture of the orientation catheter. When selecting the first aperture, the physician may position the distal end of the re-entry device at a longitudinal position (i.e., a position along the longitudinal axis of the orientation catheter) that is in general alignment with the first aperture. The physician may then rotate the re-entry device until the distal end of the re-entry device enters the first aperture. Once the distal end of the re-entry device has entered the first aperture, the re-entry device may be advanced through the first aperture. Similarly, when selecting the second aperture, the physician may position the distal end of the re-entry device at a longitudinal position that is in general alignment with the second aperture. The physician may then rotate the re-entry device until the distal end of the re-entry device enters the second aperture. The re-entry device may then be advanced through the second aperture.

If the re-entry device is subjected to plastic bending during the procedure, then the physicians ability to selectively access the first aperture and/or the second aperture can be destroyed or impaired. This is because a bent wire extending through a curved wire lumen will seek a single preferred orientation relative to the wire lumen. When the wire assumes the preferred orientation, the wire will be oriented so that a curvature plane defined by the longitudinal axis of the wire is coplanar with a curvature plane defined by the longitudinal axis of the wire lumen. In this orientation, the bend in the wire generally follows the curved path of the wire lumen and elastic deflection of the wire is at a minimum.

When the wire has been plastically bent, there is no one-to-one correspondence between rotational movement at the proximal end of the wire and rotational movement at the distal end of the wire. As the proximal end of the wire is rotated, torsional stress and/or strain builds in the wire, but the bent portion of the wire continues to assume the single preferred orientation. This condition persists until the torsional stress in the wire becomes large enough to overcome the bent wire's tendency to remain in the preferred orientation. At this point, the wire quickly rotates one complete revolution so that the bent portion of the wire again assumes the preferred orientation. The wire leaps past all other orientations so that the physician is not able to seek out an orientation that will allow him or her to insert the distal end of the re-entry device into a selected aperture of the orienting catheter.

Exemplary therapy systems disclosed in this detailed description may include provisions to avoid plastic bending of the re-entry device. More particularly, the wire of the re-entry device may include a strengthened region that makes the wire more resistant to plastic deformation during bending. The strengthened region of the wire may be configured so that the wire can be bent to conform with a tortuous path without plastic deformation so that the wire elastically recovers. In some useful embodiments, the bend radius is greater than about 0.2 inches and less than about 1.0 inches. In some particularly useful embodiments, the bend radius is greater than about 0.4 inches and less than about 0.8 inches. A wire that can be bent by a bend radius within this useful range without plastic deformation will provide physicians with the ability to select between the first aperture and the second aperture while the wire is extending through the tortuous paths found in the human vasculature.

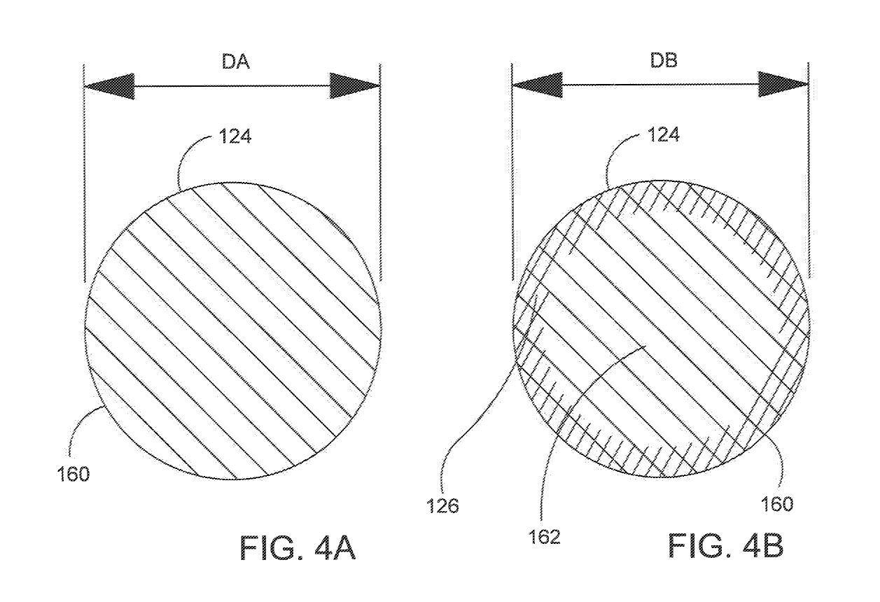

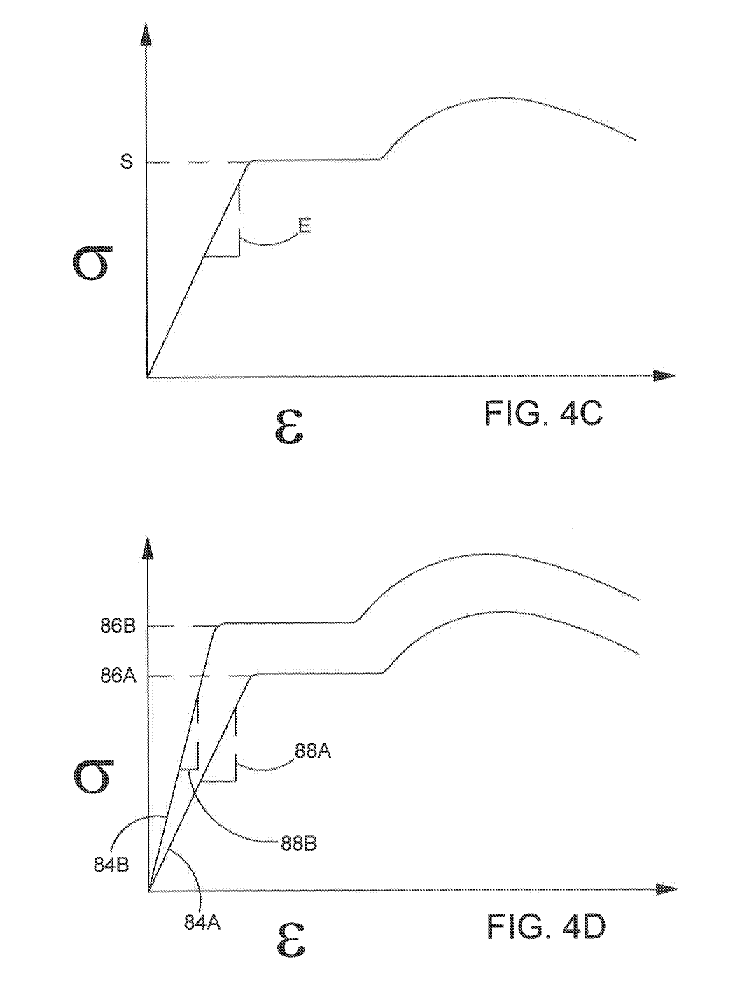

FIG. 4A and FIG. 4B are stylized cross-sectional views schematically illustrating the structure of a wire 160. FIG. 4A schematically illustrates wire 160 prior to processing in accordance with this detailed description. FIG. 4B schematically illustrates wire 160 after processing in accordance with this detailed description. FIG. 4A and FIG. 4B may be collectively referred to as FIG. 4. With reference to FIG. 4, it will be appreciated that wire 160 of FIG. 4B includes a strengthened region 126.

In the embodiment of FIG. 4, processing has produced a change in the outer diameter of wire 160. Prior to processing, wire 160 has a diameter DA that is illustrated with dimension lines in FIG. 4A. After processing, wire has a diameter of DB that is illustrated with dimension line in FIG. 4B. Diameter DB is slightly smaller than diameter DA in the embodiment of FIG. 4.

Various methods may be used to create the strengthened region 126 in the wire of FIG. 4B without deviating from the spirit and scope of this detailed description. Examples of processes that may be used to create a strengthened region include heat treating, case hardening, peening, burnishing, coining, cold working, strain hardening and work hardening. Examples of peening processes that may be used to create a strengthened region include shot peening and laser shock peening.

Strengthened region 126 is illustrated using crosshatched lines in FIG. 4B. With reference to FIG. 4B, it will be appreciated that strengthened region 126 has a generally annular shape in which strengthened region 126 encircles a central region 162 of wire 160. Strengthened region 126 may be created, for example, when a cold working process plastically deforms material near outer surface 124 of wire 160. The cold working process may apply pressure to material near outer surface 124 with a level of intensity sufficient to induce plastic deformation in a region of material near outer surface 124. The plastic deformation produced during the cold working process may work harden the material to create strengthened region 126 (schematically illustrated in FIG. 4B) while central portion 162 retains a relatively high level of toughness and ductility.

Wire 160 may comprise various materials without deviating from the spirit and scope of this detailed description. Examples of materials that may be suitable in some applications include stainless steel and nitinol. For a number of years, commercially available grades of stainless steel have been designated using a numerical index system created by the American Iron and Steel Institute (AISI) and the Society of Automotive Engineers (SAE). Commercially available grades of stainless steel that may be suitable in some applications include 301, 302, 304, and 316. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL). In some embodiments, nitinol alloys can include in the range of about 50 to about 60 weight percent nickel, with the remainder being essentially titanium. Within the family of commercially available nitinol alloys, are categories designated as “superelastic” (i.e. pseudoelastic) and “linear elastic” which, although similar in chemistry, exhibit distinct and useful mechanical properties.

FIG. 4C is an exemplary stress-strain diagram. Stress-strain diagrams are graphical tools commonly used to understand and/or explain the mechanical behavior of engineering materials. Each stress-strain diagram includes a graph showing stress plotted as a function of strain. Stress-strain diagrams are typically created with data collected using a tensile testing machine. Tensile testing machines that may be used to create stress-strain diagrams are commercially available from MTS Systems Corporation (Eden Prairie, Minn., USA) and Instron Corporation (Norwood, Mass., USA). Tensile testing is typically performed on material specimens that are fabricated to standard dimensions which are compatible with the tensile testing machine being used. The specimen is mounted in the grips of the test machine and subjected to tensile stress as the resulting strain is measured. Specimens of this type are tested in tensile testing machines to gain an understanding of the way a particular material will behave when placed under stress (e.g., stress due to bending).

Various material properties can be illustrated using a stress-strain diagram. These material properties include the elastic limit and the elastic modulus of the material. All materials have an elastic limit beyond which stress will cause permanent changes to the material. When a brittle material (e.g., glass) is stressed beyond its elastic limit the material will shatter. When a ductile material (e.g., steel) is stressed beyond its elastic limit plastic deformation of the material will typically occur. An elastic limit S is shown on the stress-strain diagram of FIG. 4C. At stresses below the elastic limit, the material will respond elastically (e.g., the material will return to its original shape when the external forces acting on it are removed). The elastic modulus E of the material may be represented by the slope of a linear portion of the stress-strain plot. In this portion of the plot, the material is being elastically deformed and the strain is proportional to the applied stress.

FIG. 4D is a stress-strain diagram illustrating changes occurring in the mechanical behavior of the material in a strengthened region of a wire that has been processed in accordance with this detailed description. A first stress-strain plot 84A and a second stress-strain plot 84B shown in the diagram of FIG. 4D. First stress-strain plot 84A represents the behavior of the material before processing and second stress-strain plot 84B represents the behavior of the material after processing.

With reference to FIG. 4D, it will be appreciated that the processing has caused a change in the behavior of the material in the strengthened region. Before processing the material has a first elastic limit 86A and after processing the material has a second elastic limit 86B. With reference to FIG. 4D, it will be appreciated that second elastic limit 86B is greater than first elastic limit 86A. When this is the case, the material has greater resistance to plastic deformation. Before processing, the material has a first elastic modulus 88A, and after processing, the material has a second elastic modulus 88B. With reference to FIG. 4D, it will be appreciated that the slope corresponding to second elastic modulus 88B is steeper than the slope corresponding to first elastic modulus 88A. It should be noted that the stress-strain plots of FIG. 4D are provided for purposes of illustration only and are not intended to be taken as representing data from a specific example or experiment.

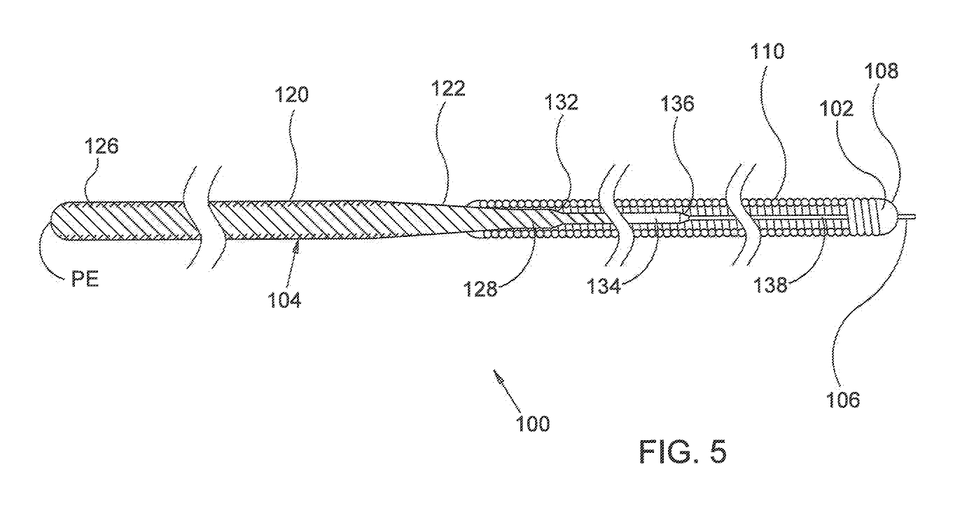

FIG. 5 is cross-sectional view showing a re-entry device 100 in accordance with the present detailed description. Re-entry device 100 comprises a tip member 102 that is fixed to a core wire 104. A coil 110 is disposed about a distal portion of core wire 104 in the embodiment of FIG. 5. Core wire 104 of FIG. 5 comprises a proximal segment 120 that extends between a proximal end PE and a first tapered segment 122. In the embodiment of FIG. 5, coil 110 extends between first tapered segment 122 and tip member 102.

A first intermediate segment 128 of core wire 104 extends between first tapered segment 122 and a second tapered segment 132. A second intermediate segment 134 of core wire 104 extends between second tapered segment 132 and a third tapered segment 136. A distal segment 138 of core wire 104 extends between third tapered segment 136 and tip member 102. With reference to FIG. 5, it will be appreciated that tip member 102 is fixed to distal segment 138 of core wire 104.

In the embodiment of FIG. 5, a probe 106 of re-entry device 100 extends distally beyond a distal surface 108 of tip member 102. In some embodiments, probe 106 comprises a portion of distal segment 138 extending beyond distal surface 108. In other embodiments, probe 106 and tip member 102 are formed from a single piece of material. When this is the case, various manufacturing processes may be used to fabricate tip member 102 and probe 106. The tip member and probe may be formed, for example, using manufacturing processes such as, for example, casting and molding. The tip member may also be fabricated by manufacturing processes that remove material from a piece of stock material to produce a desired profile. Examples of processes that may be used to remove material from a piece of stock material include grinding and machining (e.g., turning on a lathe).

Proximal segment 120 of core wire 104 comprises a strengthened region 126 that is illustrated using crosshatched lines in FIG. 5C. Core wire 104 may be fabricated by cold working a wire to produce strengthened region 126 along the length of the wire. A centerless grinding process may be used to create first tapered segment 122, first intermediate segment 128, second tapered segment 132, second intermediate segment 134, third tapered segment 136, and distal segment 138. With reference to FIG. 5, it will be appreciated that strengthened region 126 terminates at a location proximal of coil 110.

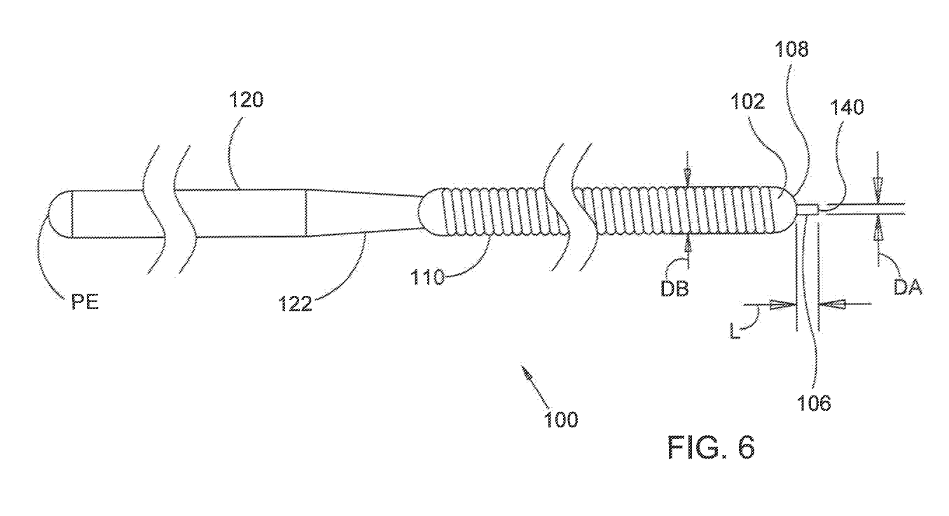

FIG. 6 is an enlarged plan view further illustrating re-entry device 100 shown in the previous figure. Re-entry device 100 comprises a tip member 102 having a distal surface 108. In the embodiment of FIG. 6, distal surface 108 of tip member 102 has a generally convex shape. In some cases, tip member 102 may have a generally hemispherical shape. A probe 106 of re-entry device 100 extends distally beyond distal surface 108. Probe 106 terminates at a distal face 140. In FIG. 6, distal face 140 is shown as a straight line representing a substantially flat surface. With reference to FIG. 6, it will be appreciated that distal face 140 is substantially perpendicular to the longitudinal axis of probe 106.

A number of exemplary dimensions associated with probe 106 are illustrated in FIG. 6. In the embodiment of FIG. 6, probe 106 extends beyond distal surface 108 of tip member 102 by a distance L. Also in the embodiment of FIG. 6, probe 106 has a diameter DA and tip member 102 has a diameter DB. With reference to FIG. 6, it will be appreciated that diameter DB of tip member 102 is generally greater than diameter DA of probe 106.

In some useful embodiments, diameter DA of probe 106 is between about 0.0020 inches and about 0.0055 inches. In some useful embodiments, diameter DB of tip member 102 is between about 0.008 inches and about 0.035 inches. In some useful embodiments, length L of probe 106 is between about 0.003 inches and about 0.032 inches. In FIG. 6, a coil 110 is shown extending between tip member 102 and a first tapered segment 122. Core wire 104 comprises a proximal segment 120 that extends between a proximal end PE and a first tapered segment 122.

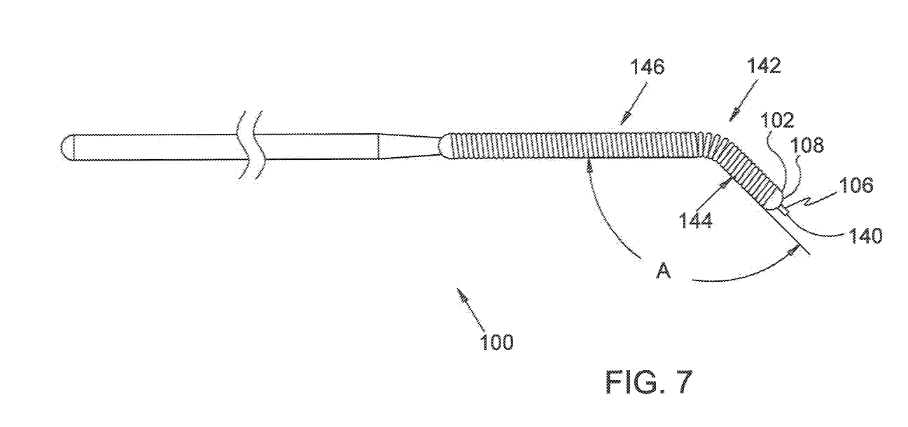

FIG. 7 is a plan view of an exemplary re-entry device 100 in accordance with the present detailed description. Re-entry device 100 comprises a tip member 102 having a distal surface 108. In the embodiment of FIG. 7, distal surface 108 of tip member has a generally convex shape. In some cases, tip member 102 may have a generally hemispherical shape. A probe 106 of re-entry device 100 extends distally beyond distal surface 108. Probe 106 terminates at a distal face 140. In FIG. 7, distal face 140 is shown as a straight line representing a substantially flat surface.

In FIG. 7, re-entry device 100 is shown being bent at an angle A. Accordingly, it can be said that re-entry device 100 includes a bend 142. In some useful embodiments of re-entry device 100, angle A is between about 90 degrees and about 180 degrees. In some particularly useful embodiments of re-entry device 100, angle A is between about 120 degrees and about 150 degrees. Re-entry device 100 has a distal leg 144 disposed distally of bend 142 and a proximal leg 146 disposed proximally of bend 142.

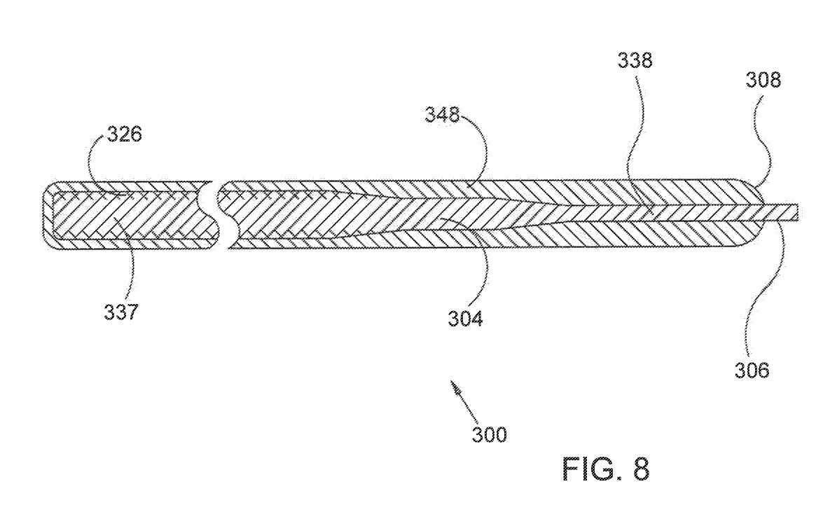

FIG. 8 is a cross-sectional view of an exemplary re-entry device 300 in accordance with the present detailed description. Re-entry device 300 comprises a core wire 304 and a jacket 348 that is disposed about a portion of core wire 304. Core wire 304 comprises a distal segment 338 and a proximal segment 337. Proximal segment 337 of core wire 304 comprises a strengthened region 326 that is illustrated using crosshatched lines in FIG. 8. Jacket 348 terminates at a distal surface 308. A probe 306 of re-entry device 300 extends distally beyond distal surface 308. In the embodiment of FIG. 8, probe 306 comprises a portion of distal segment 338 extending beyond distal surface 308.

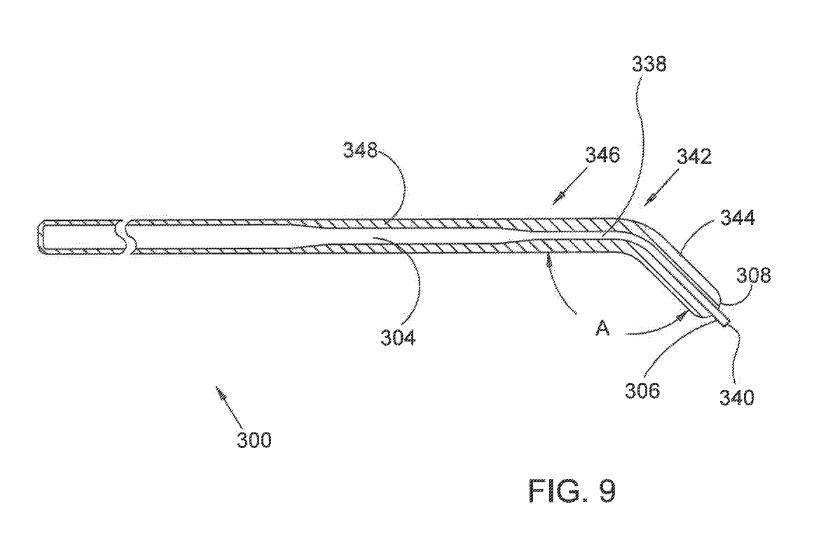

FIG. 9 is a cross-sectional view of an exemplary re-entry device 300 in accordance with the present detailed description. Re-entry device 300 comprises a core wire 304 and a jacket 348 that is disposed about a portion of core wire 304. With reference to FIG. 9, it will be appreciated that re-entry device 300 includes a bend 342 near its distal end. Re-entry device 300 has a distal leg 344 disposed distally of bend 342 and a proximal leg 346 disposed proximally of bend 342. Distal leg 344 and proximal leg 346 define an angle A. In some useful embodiments of re-entry device 300, angle A is between about 90 degrees and about 180 degrees. In some particularly useful embodiments of re-entry device 300, angle A is between about 120 degrees and about 150 degrees. Jacket 348 of re-entry device 300 terminates at a distal surface 308. A probe 306 of re-entry device 300 extends distally beyond distal surface 308. In the embodiment of FIG. 9, probe 306 comprises a distal segment 338 of core wire 304.

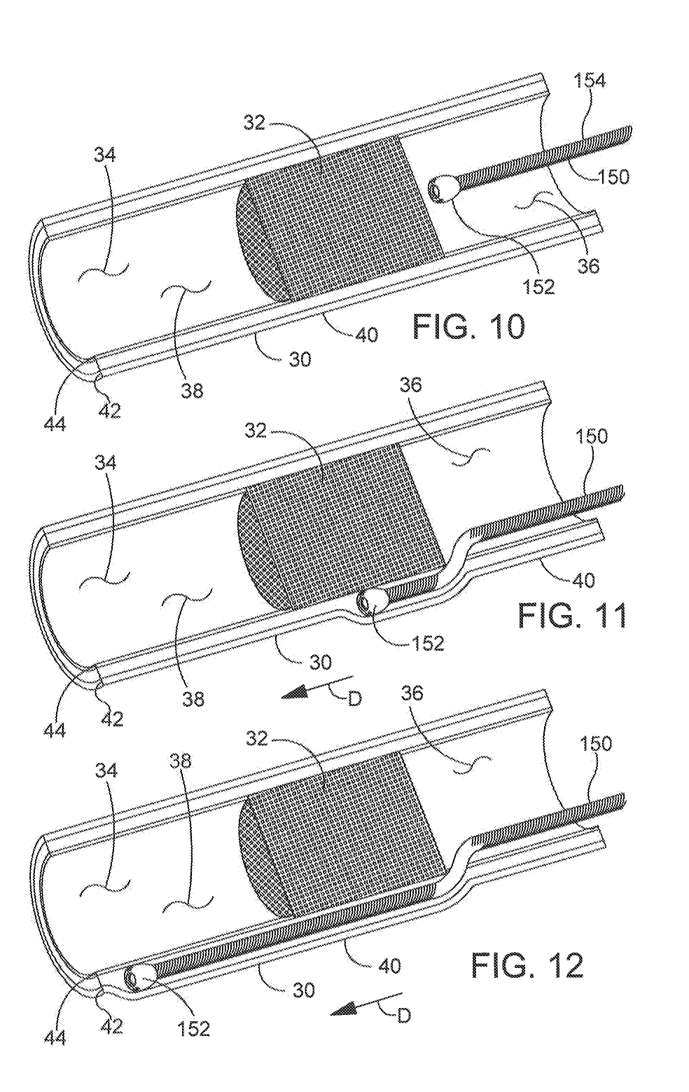

FIG. 10 through FIG. 20 are a series of stylized pictorial views illustrating various steps that may be included as part of the methods in accordance with this detailed description. The apparatus described above may be useful, for example, when performing these methods.

FIG. 10 is a longitudinal cross-sectional view of a blood vessel 30 having an occlusion 32 blocking the true lumen 34 thereof. Occlusion 32 divides true lumen 34 into a proximal segment 36 and a distal segment 38. In FIG. 10, a distal portion of a crossing device 150 is shown extending into proximal segment 36 of true lumen 34. A distal portion of crossing device 150 can be seen residing in proximal segment 36 of true lumen 34. Crossing device 150 may be advanced over a guidewire to the position shown in FIG. 10. In the embodiment of FIG. 10, crossing device 150 comprises a tip 152 that is fixed to a distal end of a shaft 154.

FIG. 11 is an additional longitudinal cross-sectional view of blood vessel 30. By comparing FIG. 11 with the previous figure, it will be appreciated that tip 152 of crossing device 150 has been advanced in a distal direction D. Distal direction D is illustrated using an arrow in FIG. 11. In the embodiment of FIG. 11, tip 152 of crossing device 150 is disposed in a position between occlusion 32 and adventitia 42 of blood vessel wall 40. Tip 152 is shown disposed adjacent occlusion 32 in FIG. 11. With reference to FIG. 11, it will be appreciated that tip 152 extends through intima 44 to the position between occlusion 32 and adventitia 42 of blood vessel 30.

FIG. 12 is an additional view of blood vessel 30 and crossing device 150 shown in the previous figure. In the embodiment of FIG. 12, tip 152 of crossing device 150 has been advanced in distal direction D so that tip 152 is disposed at a location distal of occlusion 32. In the embodiment of FIG. 12, crossing device has moved in distal direction D between intima 44 and adventitia 42 as it has advanced distally beyond occlusion 32.

With reference to the sequence of three figures described immediately above, it will be appreciated that methods in accordance with the present detailed description may include the step of advancing a crossing device along a blood vessel to a location near an occlusion. The crossing device may be advanced over a guidewire the has been previously advanced to that location. These methods may also include the step of advancing the distal end of a crossing device (e.g., crossing device 150) between an occlusion and the adventitia of a blood vessel. The crossing device may be advanced beyond the occlusion to establish a blood flow path between a proximal segment on one side of the occlusion and a distal segment on the other side of the occlusion. For example, the crossing device may spontaneously re-enter the lumen of the blood vessel as it moves past the occlusion. In some cases, the crossing device may advance distally between the intima and the adventica of the blood vessel. As the tip of the crossing device moves in a distal direction between the intima and the adventitia, the tip may cause blunt dissection of the layers forming the wall of the blood vessel. If the tip of the crossing device does not spontaneously enter the lumen, a therapy system in accordance with this detailed description may be used to pierce the intima and re-enter the lumen of the blood vessel.

In some useful methods in accordance with this detailed description, the crossing device may be rotated about its longitudinal axis and moved in a direction parallel to its longitudinal axis simultaneously. When this is the case, rotation of the crossing device may reduce resistance to the axial advancement of the crossing device. These methods take advantage of the fact that the kinetic coefficient of friction is usually less than the static coefficient of friction for a given frictional interface. Rotating the crossing device assures that the coefficient of friction at the interface between the crossing device and the surrounding tissue will be a kinetic coefficient of friction and not a static coefficient of friction.

Rotation of the crossing device can be achieved by rolling a handle portion of the crossing device between the thumb and forefinger of one hand. Two hands may also be used to rotate the crossing device. In some useful methods in accordance with this detailed description, the crossing device is rotated at a rotational speed of between about 2 revolutions per minute and about 200 revolutions per minute. In some particularly useful methods in accordance with this detailed description, the crossing device is rotated at a rotational speed of between about 50 revolutions per minute and about 150 revolutions per minute. The crossing device may be rotated at a rotational speed that is sufficient to assure that the coefficient of friction at the interface between the crossing device and the surrounding tissue will be a kinetic coefficient of friction and not a static coefficient of friction. It is contemplated that a mechanical device (e.g., an electric motor) may be used to rotate the crossing device.

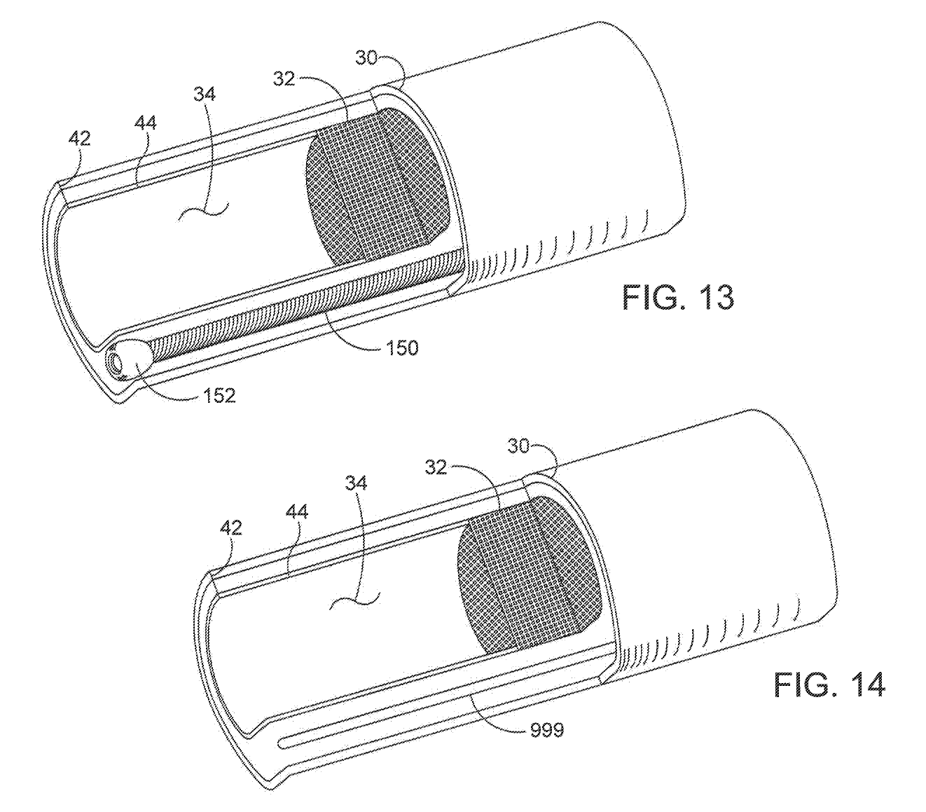

FIG. 13 is an additional stylized pictorial view of blood vessel 30 and crossing device 150 shown in the previous figure. In the embodiment of FIG. 13, tip 152 of crossing device 150 is disposed at a location distal of occlusion 32. With reference to FIG. 13, it will be appreciated that tip 152 is located between the intima 44 and the adventitia 42 of blood vessel 30.

FIG. 14 is an additional stylized pictorial view of blood vessel 30 shown in the previous figure. By comparing FIG. 14 with the previous figure, it will be appreciated that a guidewire 999 remains in the position formerly occupied by crossing device 150. Crossing device 150 has been withdrawn from blood vessel 30 while guidewire 999 has remained in the position shown in FIG. 14. The position of guidewire 999 shown in FIG. 14 may be achieved, for example, by first placing crossing device 150 in the position shown in the previous figure, then advancing guidewire 999 through a lumen defined by shaft 154 of crossing device 150. Alternately, guidewire 999 may be disposed within the lumen of shaft 154 while crossing device 150 is advanced beyond occlusion 32. With guidewire 999 in the position shown in FIG. 14, guidewire 999 may be used to direct other endovascular devices between occlusion 32 and adventitia 42. Examples of endovascular devices that may be advanced over guidewire 999 include balloon catheters, atherectomy catheters, and stent delivery catheters.

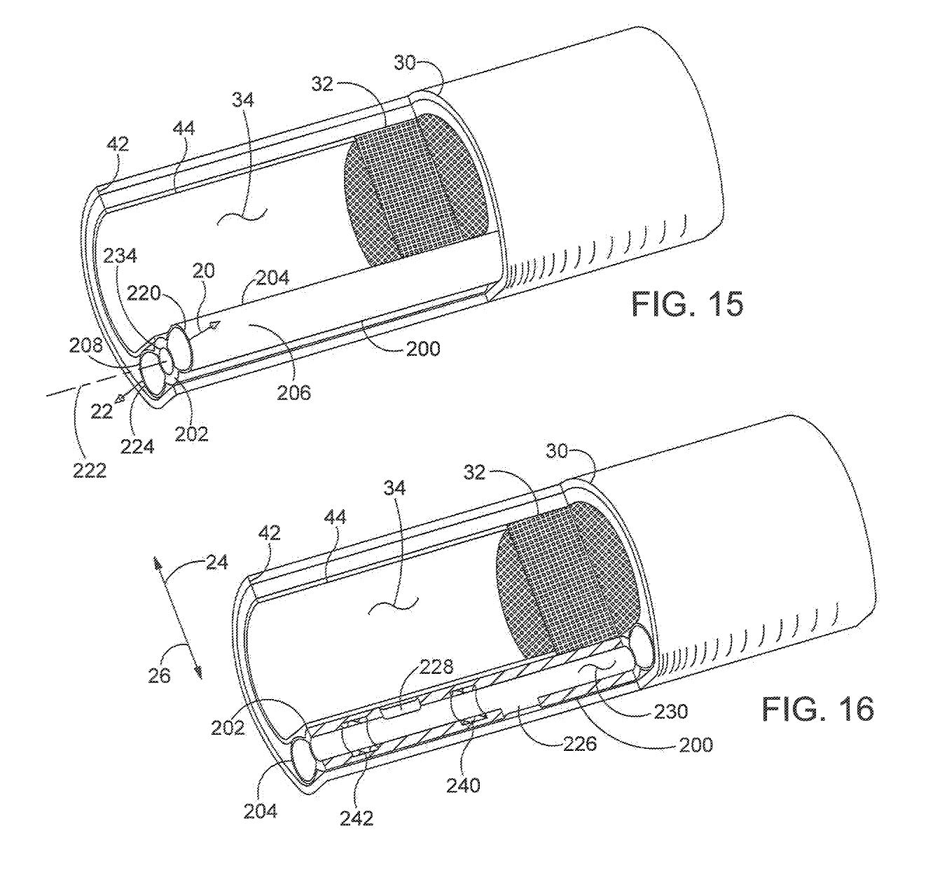

FIG. 15 is an additional stylized pictorial view of blood vessel 30 shown in the previous figure. In FIG. 15, an orienting catheter 200 is shown residing in the location previously occupied by guidewire 999. Orienting catheter 200 may be advanced into the position shown in FIG. 15, for example, by advancing orienting catheter 200 over guidewire 999 shown in the previous figure. Orienting catheter 200 comprises a shaft assembly 202 and an orienting element 204 that is carried by shaft assembly 202. Orienting element 204 is capable of assuming both a collapsed shape and an expanded shape. Orienting element 204 may be selectively placed in the collapsed shape, for example, while the orienting element is being advanced past an occlusion (e.g., occlusion 32 shown in FIG. 15). Orienting element 204 may be selectively placed in the expanded shape, for example, while the orienting catheter 200 is being used to direct a re-entry device toward the lumen of a blood vessel. In FIG. 15, orienting element 204 is shown assuming the expanded shape.

Orienting element 204 of orienting catheter 200 comprises a first portion 206 and a second portion 208. In the embodiment of FIG. 15, first portion 206 of orienting element 204 comprises a first inflatable member 220. Second portion 208 of orienting element 204 comprises a second inflatable member 224 in the embodiment of FIG. 15. First inflatable member 220 of orienting element 204 extends in a first direction 20 away from longitudinal axis 222 of shaft assembly 202. Second inflatable member 224 of orienting element 204 extends away from longitudinal axis 222 of shaft assembly 202 in a second direction 22 that is generally opposite the first direction. Shaft assembly 202 defines a distal port 234, a proximal port (not shown in FIG. 15) and a central lumen extending between the distal port and the proximal port.

FIG. 16 is an additional stylized pictorial view of blood vessel 30 and orienting catheter 200 shown in the previous figure. For purposes of illustration, orienting catheter 200 is shown in cross-section in FIG. 16. With reference to FIG. 16, it will be appreciated that guidewire 999 has been withdrawn from a central lumen 230 of orienting catheter 200. Orienting catheter 200 comprises a shaft assembly 202 defining a first aperture 226 and a second aperture 228. In the embodiment of FIG. 16, first aperture 226 extends away from central lumen 230 in a third direction 24. Second aperture 228 extends away from central lumen 230 in a fourth direction 26 that is illustrated using an arrow in FIG. 16. Third direction 24 is also represented with an arrow in FIG. 16. In the embodiment of FIG. 16, third direction 24 and fourth direction 26 extend in generally opposite directions. In FIG. 16, the arrows representing third direction 24 and fourth direction 26 are directed about 180 degrees away from one another.

Orienting catheter 200 includes an orienting element 204 that is carried by shaft assembly 202. Orienting element 204 is shown assuming an expanded shape in FIG. 16. Orienting element 204 is also capable of assuming a collapsed shape. Orienting element 204 is dimensioned such that, when the orienting element assumes an expanded shape within the blood vessel wall, the shaft assembly will assume an arbitrary one of two possible orientations relative to the blood vessel lumen. The two possible orientations comprise a first orientation and a second orientation. In the exemplary embodiment of FIG. 16, first aperture 226 is positioned so as to face the blood vessel lumen when shaft assembly 202 assumes the first orientation within the blood vessel wall. Second aperture 228 is positioned so as to face the blood vessel lumen when shaft assembly 202 assumes the second orientation within the blood vessel wall. In the embodiment of FIG. 16 orienting catheter 200 is oriented so that second aperture 228 opens toward intima 44 of blood vessel 30 and first aperture 226 opens away from intima 44. Therefore, it will be appreciated that orienting catheter 200 is assuming the second orientation in the exemplary embodiment of FIG. 16.

In the embodiment of FIG. 16, first aperture 226 and second aperture 228 are longitudinally separated from one another. Orienting catheter 200 includes a first radiopaque marker 240 that is located between first aperture 226 and second aperture 228. A second radiopaque marker 242 of orienting catheter 200 is located distally of second aperture 228.

In FIG. 16, an occlusion 32 is shown blocking lumen 34 of blood vessel 30. Occlusion 32 prevents blood from flowing through blood vessel 30. Fluid communication between a proximal segment of blood vessel lumen 34 and a distal segment of blood vessel lumen 34 may be achieved by re-entering the lumen with a re-entry device. Orienting catheter 200 may be used to direct a re-entry device toward true lumen 34 to complete a blood flow path extending around occlusion 32.

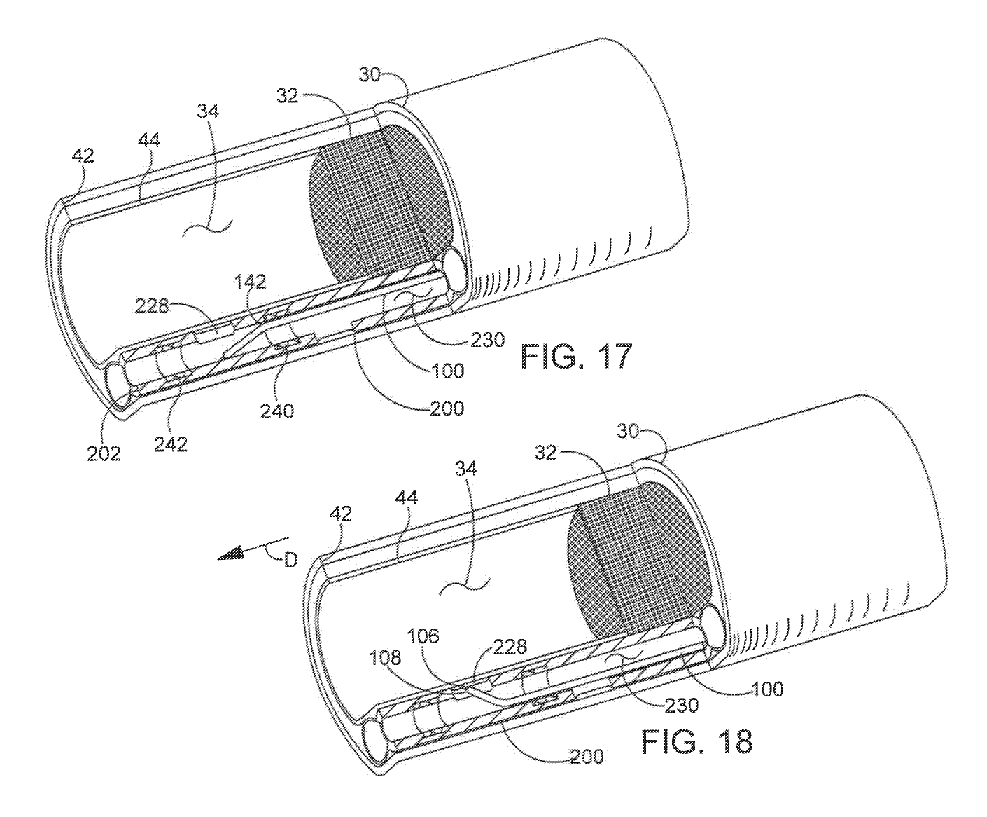

FIG. 17 is an additional stylized pictorial view of blood vessel 30 and orienting catheter 200 shown in the previous figure. In the embodiment of FIG. 17, a re-entry device 100 has been advanced into central lumen 230 of orienting catheter 200. With reference to FIG. 17, it will be appreciated that re-entry device 100 includes a bend 142. In the embodiment of FIG. 17, re-entry device 100 is biased to assume a bent shape. Also in the embodiment of FIG. 17, the wall of shaft assembly 202 is holding re-entry device 100 in a somewhat deflected state. When this is the case, re-entry device 100 can be inserted through second aperture 228 by positioning the distal end of re-entry device 100 over second aperture 228 and allowing bend 142 to assume it's natural state (i.e., bent at a sharper angle). In the embodiment of FIG. 17, rotating re-entry device 100 within central lumen 230 of orienting catheter 200 will cause the distal end of re-entry device 100 to enter second aperture 228.

A physician may use a fluoroscopic display for guidance when placing the distal end of the re-entry device in general alignment with a selected aperture. When using fluoroscopic guidance, re-entry device 100, first radiopaque marker 240, and second radiopaque marker 242 will all be brightly displayed by the fluoroscopy system. When the physician positions the distal end of re-entry device 100 slightly proximal of first radiopaque marker 240, the physician may infer that the distal end of re-entry device 100 is at a longitudinal position (i.e., a position along longitudinal axis 222) that is in general alignment with first aperture 226. The physician may then rotate re-entry device 100 so that the distal end of re-entry device 100 enters first aperture 226. The distal end of re-entry device 100 may then be advanced through first aperture 226. The physician may observe the direction that a distal portion of re-entry device 100 travels as it passes through first aperture 226. From these fluoroscopic observations, the physician can determine whether the distal end of the re-entry device is directed toward the vascular lumen or directed away from the vascular lumen. If it is determined that the re-entry device is directed toward the vascular lumen, then the re-entry device can be advanced so that the distal end of re-entry device 100 travels through the intima to a position inside the lumen 34 of blood vessel 30. If it is determined that the re-entry device is directed away from the vascular lumen, then the re-entry device can be withdrawn from first aperture 226 so that the re-entry device is again located within orienting catheter 200. At this point, the physician may determine second aperture 228 should be used for re-entry on this particular occasion.

When the physician positions the distal end of re-entry device 100 between first radiopaque marker 240 and second radiopaque marker 242, the physician may infer that the distal end of re-entry device 100 is at a longitudinal position (i.e., a position along longitudinal axis 222) that is in general alignment with second aperture 228. The physician may then rotate re-entry device 100 so that the distal end of re-entry device 100 enters second aperture 228. The distal end of re-entry device 100 may then be advanced through second aperture 228. The physician may observe the direction that a distal portion of re-entry device 100 travels as it passes through second aperture 228. From these fluoroscopic observations, the physician can confirm that the distal end of the re-entry device is directed toward the vascular lumen. If it is confirmed that the re-entry device is directed toward the vascular lumen, then the re-entry device can be advanced so that the distal end of re-entry device 100 travels through the intima to a position inside the lumen 34 of blood vessel 30.

FIG. 18 is an additional stylized pictorial view showing re-entry device 100 and orienting catheter 200 shown in the previous figure. By comparing FIG. 18 and the previous figure, it will be appreciated that re-entry device 100 has been rotated so that a distal portion of re-entry device 100 has entered second aperture 228. With reference to FIG. 18, it will be appreciated that re-entry device 100 comprises a distal surface 108 and a probe 106 extending beyond distal surface 108. In the embodiment of FIG. 18, probe 106 of re-entry device 100 is contacting intima 44 of blood vessel 30. Re-entry device 100 is shown extending distally through central lumen 230 and second aperture 228 in the embodiment of FIG. 18. By advancing re-entry device 100 further in the distal direction D, re-entry device 100 can be advanced through second aperture 228 and through intima 44.

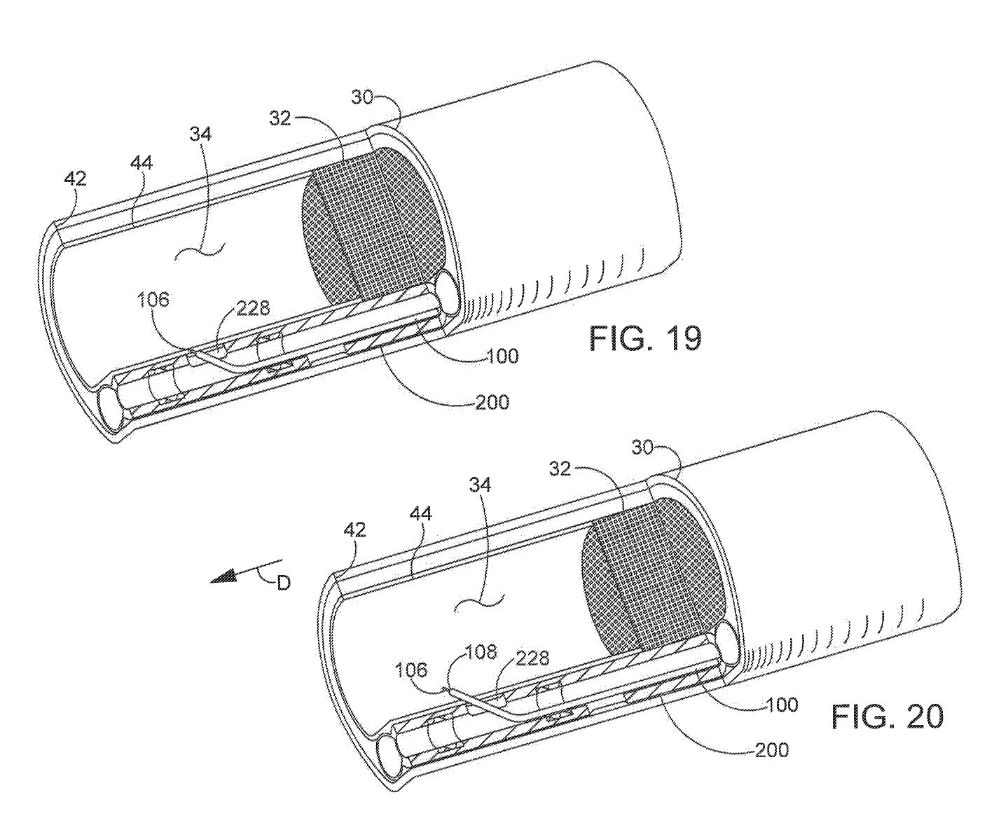

FIG. 19 is an additional stylized pictorial view showing re-entry device 100 and orienting catheter 200 shown in the previous figure. In the embodiment of FIG. 19, re-entry device 100 has been advanced further in distal direction D and probe 106 of re-entry device 100 has pierced the surface of intima 44. Probe 106 can be seen extending into intima 44 in FIG. 19. Intima 44 may be weakened when pierced by probe 106 as shown in FIG. 19. Probe 106 may also function to anchor the distal tip of re-entry device 100 to intima 44 so that the distal tip is less likely to slide along the intima when pushing forces are applied to the proximal end of re-entry device 100. The anchoring and weakening functions described above may aid a physician in advancing re-entry device 100 through intima 44.

FIG. 20 is an additional stylized pictorial view showing re-entry device 100 and orienting catheter 200 shown in the previous figure. In the embodiment of FIG. 20, a distal portion of re-entry device 100 has been advanced through intima 44. With reference to FIG. 20, it will be appreciated that distal surface 108 of re-entry device 100 is disposed in the lumen 34 of blood vessel 30. Probe 106 of re-entry device 100 can be seen extending beyond distal surface 108. Re-entry device 100 has pierced intima 44 creating a hole extending through the intima. A blood flow path extending around occlusion 32 is completed when re-entry device 100 pierces intima 44.

With particular reference to FIG. 20, it will be appreciated that a therapy system in accordance with the present detailed description may be used to establish a blood flow path around an occlusion in a blood vessel. The therapy system shown in FIG. 20 includes an orientation catheter and a re-entry device. During a procedure, the physician may selectively insert the distal end of the re-entry device into the first aperture and/or the second aperture of the orientation catheter. When selecting the first aperture, the physician may position the distal end of the re-entry device at a longitudinal position (i.e., a position along the longitudinal axis of the orientation catheter) that is in general alignment with the first aperture. The physician may then rotate the re-entry device until the distal end of the re-entry device enters the first aperture. Once the distal end of the re-entry device has entered the first aperture, the re-entry device may be advanced through the first aperture. Similarly, when selecting the second aperture, the physician may position the distal end of the re-entry device at a longitudinal position that is in general alignment with the second aperture. The physician may then rotate the re-entry device until the distal end of the re-entry device enters the second aperture. The re-entry device may then be advanced through the second aperture.

If the re-entry device is subjected to plastic bending during the procedure, then the physicians ability to selectively access the first aperture and/or the second aperture can be destroyed or impaired. This is because a bent wire extending through a curved wire lumen will seek a single preferred orientation relative to the wire lumen. When the wire assumes the preferred orientation, the wire will be oriented so that a curvature plane defined by the longitudinal axis of the wire is coplanar with a curvature plane defined by the longitudinal axis of the wire lumen. In this orientation, the bend in the wire generally follows the curved path of the wire lumen and elastic deflection of the wire is at a minimum.

When the wire has been plastically bent, there is no one-to-one correspondence between rotational movement at the proximal end of the wire and rotational movement at the distal end of the wire. As the proximal end of the wire is rotated, torsional stress and/or strain builds in the wire, but the bent portion of the wire continues to assume the single preferred orientation. This condition persists until the torsional stress in the wire becomes large enough to overcome the bent wire's tendency to remain in the preferred orientation. At this point, the wire quickly rotates one complete revolution so that the bent portion of the wire again assumes the preferred orientation. The wire leaps past all other orientations so that the physician is not able to seek out an orientation that will allow him or her to insert the distal end of the re-entry device into a selected aperture of the orienting catheter.

Exemplary therapy systems disclosed in this detailed description may include provisions to avoid plastic bending of the re-entry device. More particularly, the wire of the re-entry device may include a strengthened region that makes the wire more resistant to plastic deformation during bending that occurs as the reentry device follows a tortuous path. The strengthened region of the wire may be configured so that the wire can be bent about a bend in the tortuous path without plastic deformation. In some useful embodiments, the core wire extends through a 180 degree arc of a circle and the core wire has a centerline bend radius greater than about 0.4 inches and less than about 0.8 inches with no plastic deformation so that the core wire is able to elastically recover. A wire that can be bent to assume a bend of this type without plastic deformation will provide physicians with the ability to select between the first aperture and the second aperture while the wire is extending through the tortuous paths found in the human vasculature.

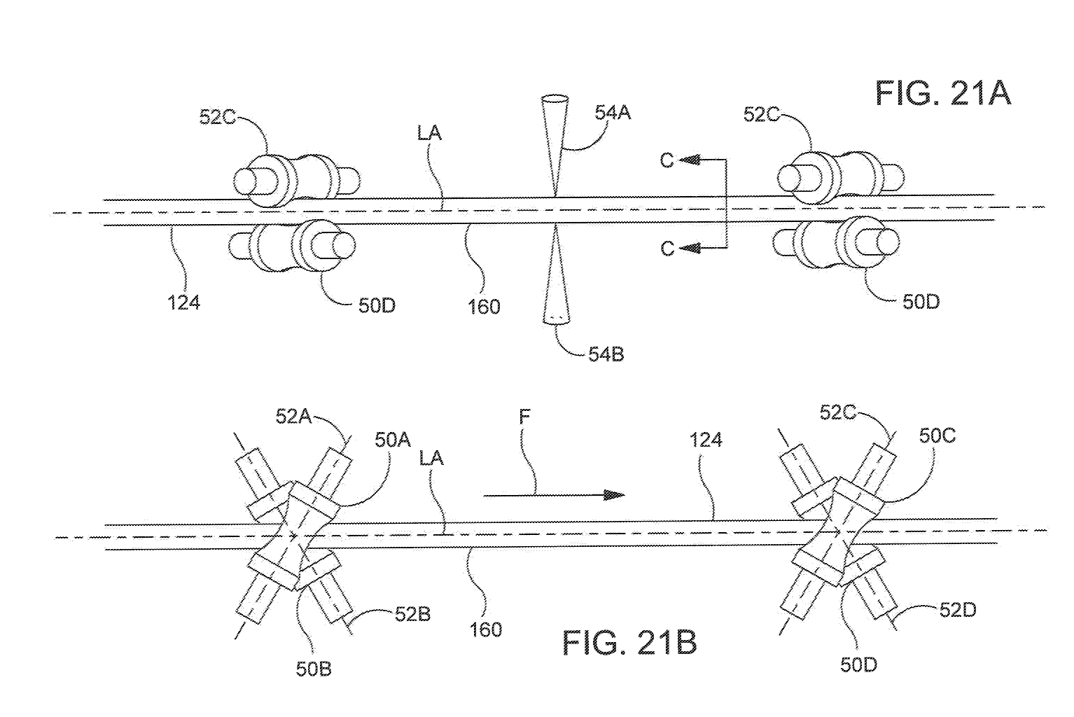

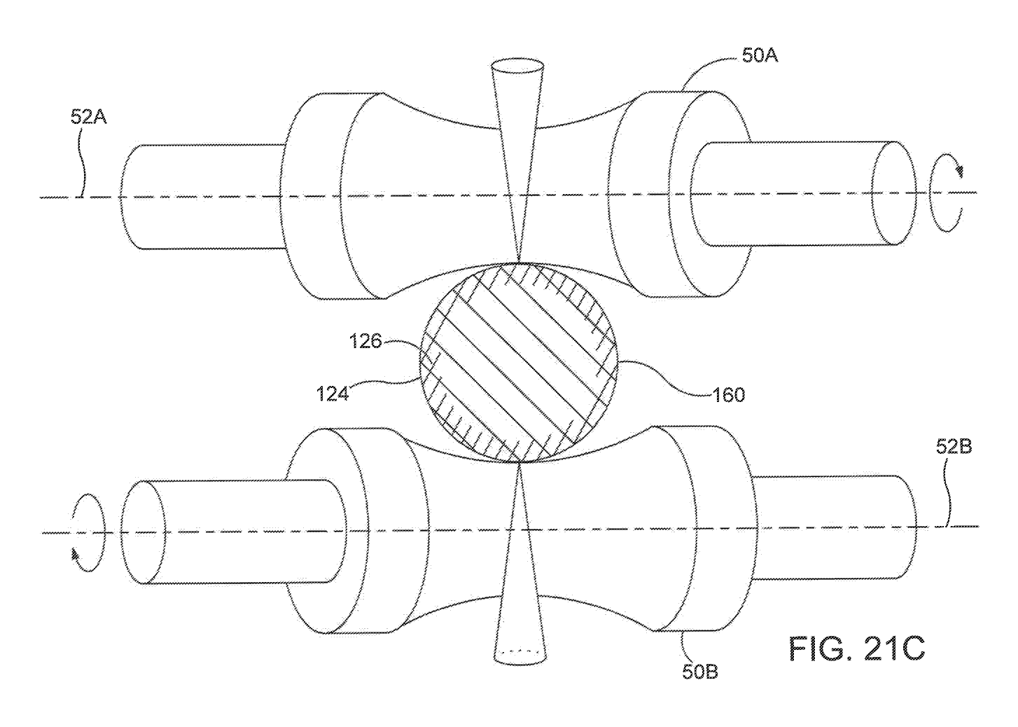

FIG. 21A, FIG. 21B, and FIG. 21C are stylized plan views illustrating an exemplary process that may be used to form a strengthened region 126 in a wire 160. FIG. 21B is taken from a viewpoint that is generally orthogonal the viewpoint used to create FIG. 21A. FIG. 21C is created from the viewpoint illustrated by section line C-C in FIG. 21A. FIG. 21A, FIG. 21B, and FIG. 21C may be collectively referred to as FIG. 21. It is customary to refer to multi-view projections using terms such as front view, top view, and side view. In accordance with this convention, FIG. 21A may be referred to as a side view of wire 160 and FIG. 21B may be referred to as a top of wire 160. The terms top view and side view are used herein as a convenient method for differentiating between the views shown in FIG. 21. It will be appreciated that the apparatus shown in FIG. 21 may be arranged in various orientations without deviating from the spirit and scope of this detailed description. Accordingly, the terms top view and side view should not be interpreted to limit the scope of the invention recited in the attached claims.

In FIG. 21, wire 160 can be seen extending between a first roller 50A and a second roller 50B. Wire 160 also extends between a third roller 50C and a fourth roller 50D in the embodiment of FIG. 21. Each of the rollers in FIG. 21 rotates about an axis of rotation. More particularly, first roller 50A rotates about an axis of rotation 52A and second roller 50B rotates about an axis of rotation 52B. Third roller 50C and a fourth roller 50D rotate about a third axis of rotation 52C and a fourth axis of rotation 52D, respectively. With particular reference to FIG. 21A, it will be appreciated that the axis of rotation of each roller is skewed relative to the longitudinal axis LA of wire 160.

In the embodiment of FIG. 21, the rotation of the rollers causes wire 160 to translate in a feed direction F. The rollers also cause wire 160 to rotate about its longitudinal axis LA as it translates. With particular reference FIG. 21B, it will be appreciated that the axis of rotation 52A of first roller 50A is skewed relative to the axis of rotation 52B of second roller 50B. The axis of rotation 52C of third roller 50C is skewed relative to the axis of rotation 52D of fourth roller 50D in the embodiment of FIG. 21B.

In the embodiment of FIG. 21, a first laser 54A directs a first series of laser pulses to strike an outer surface 124 of wire 160. As wire 160 translates and rotates relative to first laser 54A, the laser pulses produced by first laser 54A form a first helical path along outer surface 124 of wire 160. Each laser pulse striking outer surface 124 acts like a tiny peening hammer imparting a small laser spot on outer surface 124 of wire 160. Each laser pulse may impart compressive stresses into the material extending below outer surface 124 at each laser spot. A second laser 54B directs a second series of laser pulses to strike outer surface 124 of wire 160 in the embodiment of FIG. 21. As wire 160 translates and rotates relative to second laser 54B, the laser pulses produced by second laser 54B form a second helical path along outer surface 124 of wire 160. In some useful embodiments, the series of spots produced by first laser 54A and the series of spots produced second laser 54B are positioned to form a pattern of overlapping spots substantially covering the outer surface of the wire. Implementations are also possible in which spots made by a single laser form a pattern of overlapping spots substantially covering the outer surface of the wire.

FIG. 21C is an enlarged plan view created from the viewpoint illustrated by section line C-C in FIG. 21A. In FIG. 21C, wire 160 can be seen extending between first roller 50A and second roller 50B. In the embodiment of FIG. 21C, first roller 50A rotates about a first axis of rotation 52A and second roller 50B rotates about a second axis of rotation 52B. The direction that each roller rotates is illustrated using arrows in FIG. 21C. In the embodiment of FIG. 21C, the axis of rotation 52A of first roller 50A is skewed relative to the axis of rotation 52B of second roller 50B. The axis of rotation of each roller is also skewed relative to the longitudinal axis of wire 160. In the embodiment of FIG. 21C, the direction of rotation for each roller and the skewed geometric relationship between the rollers and the longitudinal axis LA of wire 160 assure that first roller 50A and second roller 50B will cause simultaneous rotation and translation of wire 160.

In the embodiment of FIG. 21, the repeated impact of laser pulses from first laser 54A and second laser 54B has produced a strengthened region 126 that is illustrated using cross-hatched lines. With reference to FIG. 4B, it will be appreciated that strengthened region 126 has a generally annular shape in which strengthened region 126 encircles a central region 162 of wire 160. In some implementations, first laser 54A and second laser 54B may be produced by a single laser beam source. Alternately, first laser 54A may be produced by a first laser beam source and second laser 54B may be produced by a second laser beam source different from the first laser beam source. Laser beam sources that may be suitable in some applications are commercially available from Trumpf Laser and Systemtechnik GmbH (Ditzingen, Del.) and Coherent, Inc. (Santa Clara, Calif., USA). Types of laser beam sources tht may be suitable in some applications include gas lasers (e.g., CO2) and solid-state lasers (e.g., ruby).

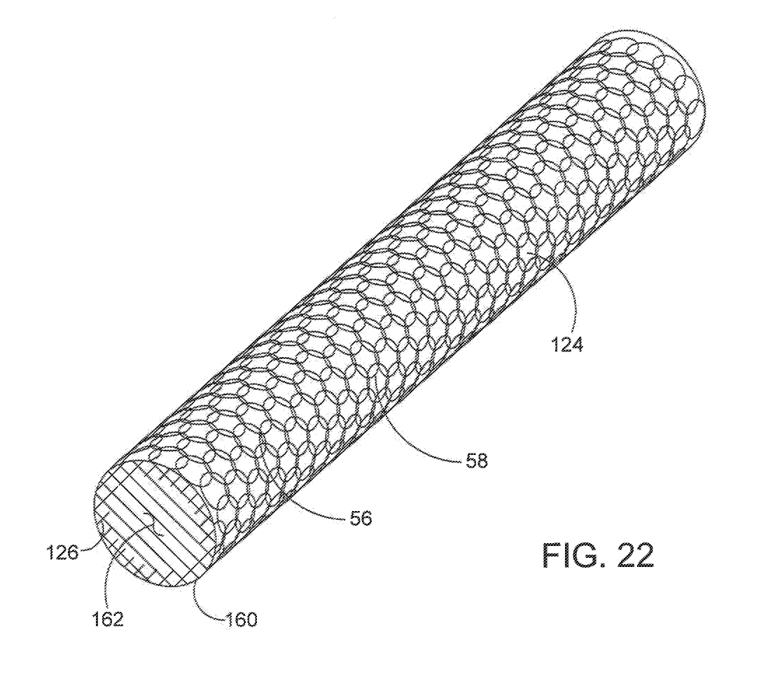

FIG. 22 is a stylized perspective view showing wire 160 shown in the previous figure. In the stylized perspective view of FIG. 22, a plurality of spots 58 can be seen on outer surface 124 of wire 160. The spots 58 shown in FIG. 22 form a pattern 56 of overlapping spots that substantially covers outer surface 124 of wire 160.

Wire 160 includes a strengthened region 126 that is illustrated using cross hatched lines in FIG. 22. In the embodiment of FIG. 22, strengthened region 126 has been produced by the repeated impact of laser pulses from first laser 54A and second laser 54B shown in the previous figure. With reference to FIG. 22, it will be appreciated that strengthened region 126 encircles a central region 162 of wire 160.

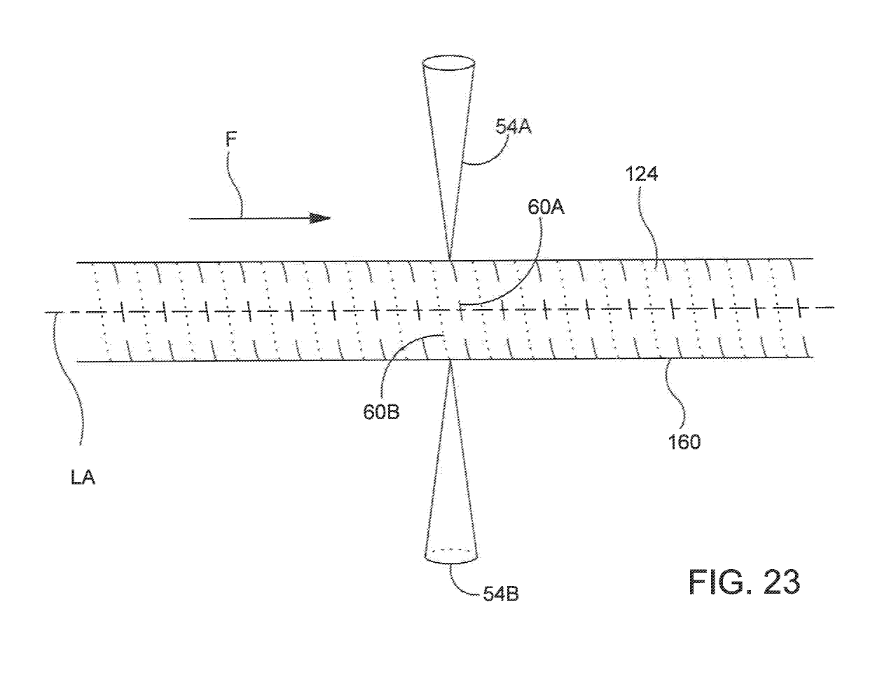

FIG. 23 is a plan view illustrating a process that may be used to form a strengthened region in a wire 160. In the embodiment of FIG. 23, wire 160 is translating in a feed direction F past a first laser 54A and a second laser 54B. Feed direction F is illustrated using an arrow in FIG. 23. In the embodiment of FIG. 23, wire 160 is rotating about its longitudinal axis LA as it translates in feed direction F.

In the embodiment of FIG. 23, first laser 54A directs a first series of laser pulses to strike an outer surface 124 of wire 160. As wire 160 translates and rotates relative to first laser 54A, the laser pulses produced by first laser 54A strike outer surface 124 along a first helical path 60A. First helical path 60A is illustrated using dashed lines in FIG. 23. Each laser pulse striking outer surface 124 acts like a tiny peening hammer imparting a small laser spot on outer surface 124 of wire 160. Each laser pulse may impart compressive stresses into the material extending below outer surface 124 at each laser spot. Second laser 54B directs a second series of laser pulses to strike outer surface 124 of wire 160 in the embodiment of FIG. 23. As wire 160 translates and rotates relative to second laser 54B, the laser pulses produced by second laser 54B strike outer surface 124 along a second helical path 60B. Second helical path 60B is illustrated using dotted lines in FIG. 23. In some useful embodiments, the series of spots produced by first laser 54A and the series of spots produced second laser 54B overlap each other to form a pattern of overlapping spots that substantially covers outer surface 124 of wire 160 (e.g., pattern 56 shown in FIG. 22).

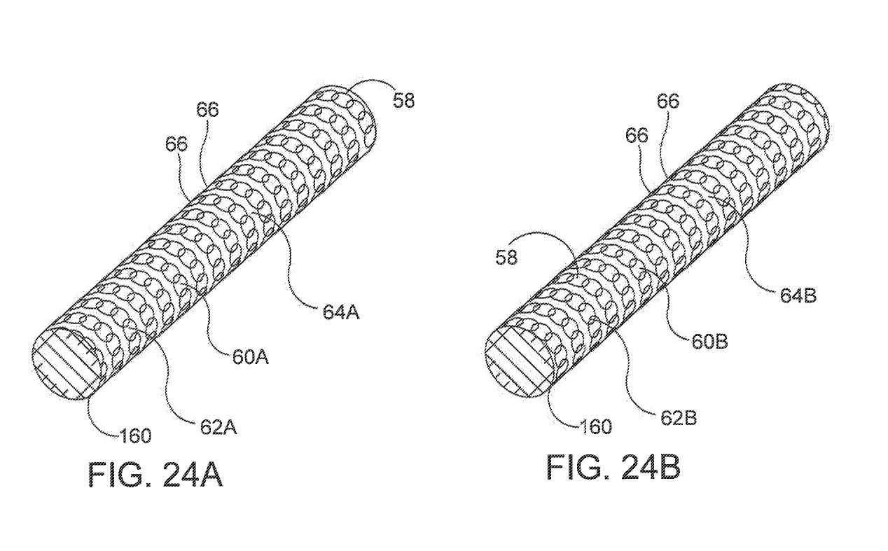

FIG. 24A is a stylized perspective view illustrating a first series 62A of overlapping laser beam spots 58 that form a first generally helical path 60A around wire 160. In the embodiment of FIG. 24A, first generally helical path 60A includes of plurality of turns 66 encircling wire 160. With reference to FIG. 24A, it will be appreciated that there is a first gap 64A between adjacent turns 66 of the overlapping laser beam spots 58 in first series 62A. FIG. 24B is a stylized perspective view illustrating a second series 62B of overlapping laser beam spots 58 that form a second generally helical path 60B around wire 160. A second gap 64B is defined by adjacent turns 66 formed by second series 62B of overlapping laser beam spots 58 as the spots follow second generally helical path 60B around wire 160.

For purposes of illustration, the first series 62A of overlapping laser beam spots 58 that form first generally helical path 60A are not shown in FIG. 24B and the second series 62B of overlapping laser beam spots 58 that form second generally helical path 60B are not shown in FIG. 24A. This allows first gap 64A and second gap 64B to be seen in FIG. 24A and FIG. 24B, respectively. In some useful embodiments, first generally helical path 60A and second generally helical path 60B are dimensioned and positioned so that the first series 62A of spots and the second series 62B of spots overlap each other. When this is the case, the spots of first series 62A and the spots of second series 62B form a pattern of overlapping spots that substantially covers outer surface 124 of wire 160. Pattern 56 shown in FIG. 22 is one example of such a pattern. In some useful embodiments, first gap 64A has a width that is less than a diameter of each spot 58. Second gap 64B may also have a width that is less than the diameter of each spot 58.

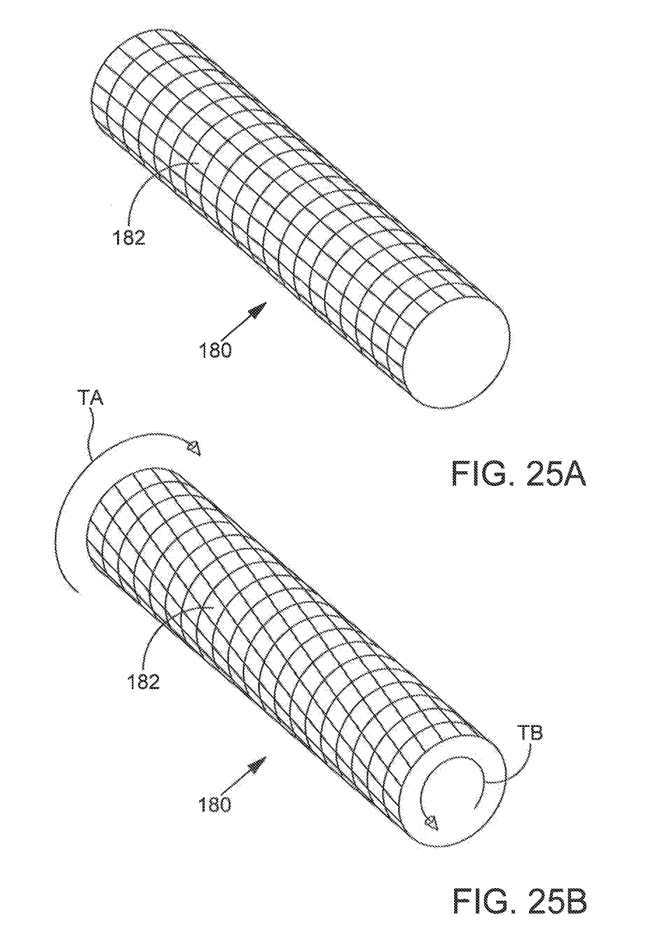

FIG. 25A is a stylized perspective view of a wire section 180. For purposes of illustration, wire section 180 is shown comprising a plurality of finite elements 182. FIG. 25B is a second stylized perspective view of wire section 180 shown in FIG. 25A. A first torque TA and a second torque TB are placing wire section 180 in torsion in the embodiment of FIG. 25B. Each torque is illustrated with an arrow in FIG. 25B.

With reference to FIG. 25B, it will be appreciated that first torque TA and second torque TB have opposite directions. In some useful embodiments, first torque TA and second torque TB apply equal and opposite moments to wire section 180. In the embodiment of FIG. 25B, first torque TA and second torque TB are cooperating to twist wire section 180. By comparing FIG. 25A and FIG. 25B, it will be appreciated that the twisting of wire section 180 has caused changes in the shapes of the finite elements 182 of wire section 180.

The twisting of wire section 180 in embodiment of FIG. 25B has caused plastic deformation in the material. In some useful methods, plastic deformation due to twisting is used to create a strengthened region in a wire. Without wishing to be bound by a particular theory of operation, it is believed that the process illustrated in FIG. 25B creates a strengthened region when dislocation movements within the grain structure of the material occur. For example, the twisting may orient the grain structure in a generally helical pattern. The atoms within the grain structure may be relocated into a configuration having a higher yield strength and a higher modulus of elasticity. For example, relatively large atoms within the grain structure may be moved against one another.

An exemplary method of processing a wire to create a strengthened region therein may now be described with reference to FIG. 25B. A method in accordance with this detailed description may include clamping a wire in appropriate fixtures and twisting the wire by creating relative rotation between the fixtures. A proximal portion of the wire and a distal portion of the wire may be clamped in a first fixture and a second fixture, respectively. Each fixture may comprise a pair of jaws that can be selectively opened and closed. The second fixture may be rotated relative to the first fixture to twist the wire.

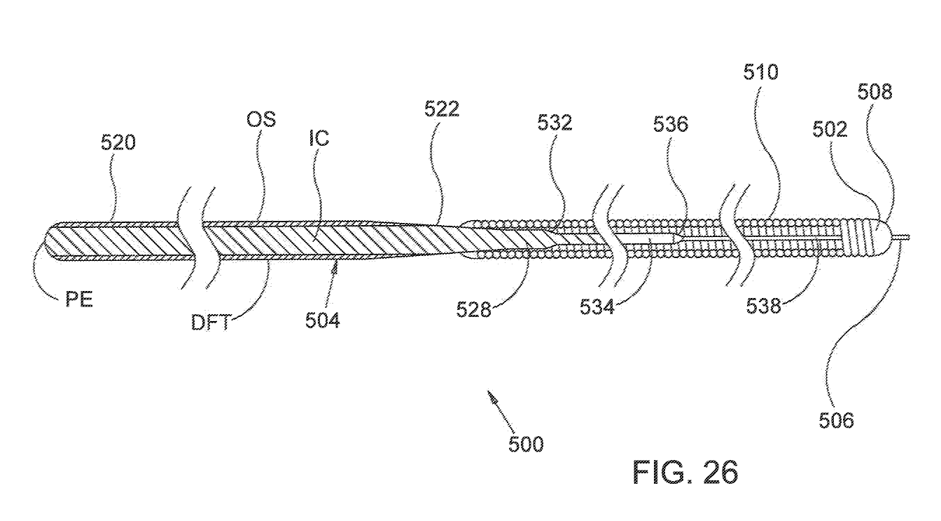

FIG. 26 is cross-sectional view showing a re-entry device 500. Re-entry device 500 comprises a tip member 502 that is fixed to a core wire 504. A coil 510 is disposed about a distal portion of core wire 504 in the embodiment of FIG. 26. Core wire 504 of FIG. 26 comprises a proximal segment 520 that extends between a proximal end PE and a first tapered segment 522. In the embodiment of FIG. 26, coil 510 extends between first tapered segment 522 and tip member 502.

A first intermediate segment 528 of core wire 504 extends between first tapered segment 522 and a second tapered segment 532. A second intermediate segment 534 of core wire 504 extends between second tapered segment 532 and a third tapered segment 536. A distal segment 538 of core wire 504 extends between third tapered segment 536 and tip member 502. With reference to FIG. 26, it will be appreciated that tip member 502 is fixed to distal segment 538 of core wire 504.

In the embodiment of FIG. 26, a probe 506 of re-entry device 500 extends distally beyond a distal surface 508 of tip member 502. In some embodiments, probe 506 comprises a portion of distal segment 538 extending beyond distal surface 508. In other embodiments, probe 506 and tip member 502 are formed from a single piece of material. When this is the case, various manufacturing processes may be used to fabricate tip member 502 and probe 506. The tip member and probe may be formed, for example, using manufacturing processes such as, for example, casting and molding. The tip member may also be fabricated by manufacturing processes that remove material from a piece of stock material to produce a desired profile. Examples of processes that may be used to remove material from a piece of stock material include grinding and machining (e.g., turning on a lathe).

Core wire 504 has a proximal portion comprising an outer sheath OS and an inner core IC. In the embodiment of FIG. 26, the proximal portion of core wire 504 comprises a length of drawn filled tube DFT. In some embodiments, outer sheath OS comprises nitinol and inner core IC comprises stainless steel. In other embodiments, outer sheath OS comprises stainless steel and inner core IC comprises nitinol. Drawn filled tube that may be suitable for some applications is commercially available from Fort Wayne Metals of Fort Wayne, Pa. With reference to FIG. 26, it will be appreciated that outer sheath OS terminates at a location proximal of coil 510. A centerless grinding process may be used to create first tapered segment 522, first intermediate segment 528, second tapered segment 532, second intermediate segment 534, third tapered segment 536, and distal segment 538. With reference to FIG. 26, it will be appreciated that the centerless grinding process used to create these elements may selectively remove portions of outer sheath OS.

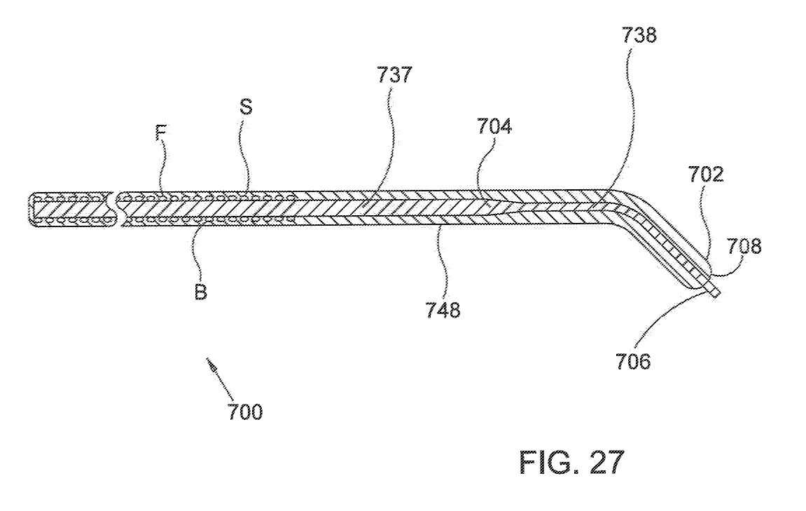

FIG. 27 is a cross-sectional view of an exemplary re-entry device 700. Re-entry device 700 comprises a core wire 704 including a distal segment 738 and a proximal segment 737. A sheath S is disposed about a portion of proximal segment 737. In the embodiment of FIG. 27, sheath S comprises a plurality of filars F that are interlinked with one another to form a hollow braid B. A jacket 748 is disposed about sheath S and a portion of core wire 704. Jacket 748 may comprise, for example, a thermoplastic material that has been extruded over core wire 704 and sheath S. The material of Jacket 748 forms a tip member 702 having a distal surface 708. A probe 706 of re-entry device 700 extends distally beyond distal surface 708. In the embodiment of FIG. 27, probe 706 comprises a portion of distal segment 738 extending beyond distal surface 708.

Methods in accordance with this detailed description may now be described with reference to the figures described above. Such methods may include creating a strengthened region in a core wire. Various processes may be used to create the strengthened region without deviating from the spirit and scope of this detailed description. Examples of processes that may be used to create a strengthened region in a wire include heat treating, case hardening, peening, burnishing, coining, cold working, strain hardening and work hardening. Examples of peening processes that may be used to create a strengthened region include shot peening and laser shock peening.

Methods in accordance with this detailed description may also include the step of assembling a reentry device. The core wire including the strengthened region may become part of a reentry device during the assembly process. The assembled re-entry device may be provided to a user (e.g., a physician). Instructions for treating a patient using the re-entry device may be provided to the user along with the re-entry device. The instructions may also be provided by the user before and/or after the re-entry device is provided to the user. The instructions may be provided in the form of an instruction sheet including text and figures. The instructions may also be provided orally (e.g., oral instructions provided during a one-on-one training session). The instructions may teach to the user how to perform various methods in accordance with this detailed description. The user may be instructed to insert the distal end of the re-entry device into a lumen defined by an orienting catheter that is extending along the blood vessel, position the distal end proximate a first aperture, and rotate the re-entry device until the distal end enters the first aperture.

With particular reference to FIG. 17 through FIG. 19, it will be appreciated that a physician may use a fluoroscopic display for guidance when placing the distal end of the re-entry device in general alignment with a selected aperture. The orienting catheter may include a first radiopaque marker and a second radiopaque marker that will be brightly displayed on a fluoroscopy display. The reentry device may also incorporate radiopaque materials. When the physician positions the distal end of the re-entry device slightly proximal of the first radiopaque marker, the physician may infer that the distal end of the re-entry device is at a longitudinal position that is in general alignment with a first aperture of the orientation catheter. The physician may then rotate the re-entry device so that the distal end of the re-entry device enters the first aperture. The distal end of re-entry device may then be advanced through the first aperture. The physician may observe the direction that a distal portion of the re-entry device travels as it passes through the first aperture. From these fluoroscopic observations, the physician can determine whether the distal end of the re-entry device is directed toward the vascular lumen or directed away from the vascular lumen. If it is determined that the re-entry device is directed toward the vascular lumen, then the re-entry device can be advanced so that the distal end of the re-entry device travels through the intima to a position inside the lumen of the blood vessel. If it is determined that the re-entry device is directed away from the vascular lumen, then the re-entry device can be withdrawn from the first aperture so that the re-entry device is again located within the orienting catheter. At this point, the physician may determine the second aperture of the orienting catheter should be used for re-entry on this particular occasion.

When the physician positions the distal end of the re-entry device between the first radiopaque marker and the second radiopaque marker, the physician may infer that the distal end of the re-entry device is at a position that is longitudinally aligned with the second aperture. The physician may then rotate the re-entry device so that the distal end of the re-entry device enters the second aperture. The distal end of the re-entry device may then be advanced through the second aperture. The physician may observe the direction that a distal portion of the re-entry device travels as it passes through the second aperture. From these fluoroscopic observations, the physician can confirm that the distal end of the re-entry device is directed toward the vascular lumen. If it is confirmed that the re-entry device is directed toward the vascular lumen, then the re-entry device can be advanced so that the distal end of the re-entry device travels through the intima to a position inside the lumen of the blood vessel.

From the foregoing, it will be apparent to those skilled in the art that the present disclosure provides, in exemplary non-limiting embodiments, devices and methods for the treatment of chronic total occlusions. Further, those skilled in the art will recognize that the present disclosure may be manifested in a variety of forms other than the specific embodiments described and contemplated herein. Accordingly, departures in form and detail may be made without departing from the scope and spirit of the present disclosure as described in the appended claims.

What is claimed is:

1. A system for treating a blood vessel including a blood vessel lumen defined by a blood vessel wall, the blood vessel lumen being at least partially obstructed, the system comprising:
a shaft assembly including an orienting element, the orienting element having an expanded shape dimensioned such that, when the orienting element assumes the expanded shape within the blood vessel wall, the shaft assembly will assume an arbitrary one of two possible orientations relative to the blood vessel lumen, the two possible orientations comprising a first orientation and a second orientation;
the shaft assembly defining a shaft lumen, a first aperture and a second aperture, the first aperture being positioned to face the blood vessel lumen when the shaft assembly assumes the first orientation and the second aperture being positioned to face the blood vessel lumen when the shaft assembly assumes the second orientation;
a re-entry device extending into the central lumen, the re-entry device comprising a core wire, the core wire being configured such that bending stresses created in the core wire during bending of the core wire to follow a tortuous path are less than the elastic limit of the core wire so that the core wire will elastically recover from the bending upon release.

2. The system of claim 1, wherein the core wire comprises a strengthened region.

3. The system of claim 2, wherein the strengthened region is produced by heat treating the core wire.

4. The system of claim 2, wherein the strengthened region is produced by case hardening the core wire.

5. The system of claim 2, wherein the strengthened region is produced by cold working the core wire.

6. The system of claim 2, wherein the strengthened region is produced by work hardening a portion of the core wire.

7. The system of claim 2, wherein the strengthened region is produced by plastically deforming the core wire.

8. The system of claim 7, wherein the strengthened region is produced by twisting the wire.

9. The system of claim 2, wherein the strengthened region is produced burnishing the wire.

10. The system of claim 2, wherein the strengthened region is produced by shot peening the wire.

11. The system of claim 2, wherein the strengthened region is produced by laser shock peening the wire.

12. The system of claim 2, wherein the strengthened region has a first depth and an annular shape.

13. The system of claim 12, wherein the strengthened region encircles a central region of the core wire.

14. The system of claim 13, wherein:
material in the strengthened region has a first elastic limit;
material in the central region has a second elastic limit; and
the first elastic limit is greater than the second elastic limit.

15. The system of claim 13, wherein:
material in the strengthened region has a first level of ductility;
material in the central region has a second level of ductility; and
the second level of ductility is greater than the first level of ductility.

16. The system of claim 2, wherein:
the core wire comprises a proximal portion and a distal portion;
the strengthened region extends along at least a portion of the proximal portion; and
the strengthened region terminates at a location proximal of the distal portion of the core wire.

17. The system of claim 16, wherein:
material in the strengthened region has a first elastic limit;
material in the distal portion has a second elastic limit; and
the first elastic limit is greater than the second elastic limit.

18. The system of claim 1, wherein:
the re-entry device comprises a tip member fixed to the core wire; and
a distal portion of the core wire extends beyond a distal surface of the tip member.

19. The system of claim 18, wherein the distal portion of the core wire extends beyond the tip member by a distance that is greater than about 0.003 inches and less than about 0.012 inches.

20. The system of claim 19, wherein the distal portion of the core wire extends beyond the tip member by a distance that is greater than about 0.004 inches and less than about 0.008 inches.

21. The system of claim 18, wherein the distal portion of the core wire has a diameter that is greater than about 0.002 inches and less than about 0.006 inches.

22. The system of claim 18, wherein the distal portion of the core wire has an aspect ratio of length to diameter that is greater than about 1.

23. The system of claim 22, wherein the distal portion of the core wire has an aspect ratio of length to diameter that is greater than about 2.

24. The system of claim 18, wherein:
the distal portion of the core wire has a maximum diameter DW;
the tip member has a maximum diameter DT; and
the maximum diameter DT is greater than the maximum diameter DW of the distal portion of the core wire.

25. The system of claim 24, wherein a ratio of the maximum diameter DT of the tip member to the maximum diameter DW of the distal portion of the core wire is greater than about 3.

26. The system of claim 18, wherein the core wire comprises a proximal leg, a distal leg, and a bend disposed between the proximal leg and the distal leg.

27. The system of claim 26, wherein the bend extends through an angular range that is greater than about 90 degrees and less than about 180 degrees.

28. The system of claim 27, wherein the bend extends through an angular range that is greater than about 120 degrees and less than about 150 degrees.

29. The system of claim 26, wherein the distal leg has a length that is greater than about 0.040 inch and less than about 0.300 inch.

30. The system of claim 1, wherein:
the core wire extends through an iliac bifurcation of an adult human patient when the core wire is following the tortuous path;
the core wire extends through a 180 degree arc of a circle when the core wire is following the tortuous path; and
the core wire has a centerline bend radius greater than about 0.2 inches and less than about 1.0 inches when the core wire is following the tortuous path.

31. The system of claim 1, wherein;
the core wire extends through a 180 degree arc of a circle when the core wire is following the tortuous path; and
the core wire has a centerline bend radius greater than about 0.4 inches and less than about 0.8 inches when the core wire is following the tortuous path.

32. A system for treating a blood vessel including a blood vessel lumen defined by a blood vessel wall, the blood vessel lumen being at least partially obstructed, the system comprising:
a shaft assembly including an orienting element, the orienting element having an expanded shape dimensioned such that, when the orienting element assumes the expanded shape within the blood vessel wall, the shaft assembly will assume an arbitrary one of two possible orientations relative to the blood vessel lumen, the two possible orientations comprising a first orientation and a second orientation;
the shaft assembly defining a shaft lumen, a first aperture and a second aperture, the first aperture being positioned to face the blood vessel lumen when the shaft assembly is assuming the first orientation and the second aperture being positioned to face the blood vessel lumen when the shaft assembly is assuming the second orientation;
a re-entry device extending into the central lumen, the re-entry device comprising a tip member fixed to a core wire, a proximal portion of the core wire comprising an sheath disposed about a core, wherein a distal portion of the core extends beyond a distal surface of the tip member.

33. The system of claim 32, wherein the proximal portion of the core wire comprises drawn filled tube.

34. The system of claim 32, wherein the sheath comprises nitinol and core comprises stainless steel.

35. The system of claim 32, wherein the sheath comprises stainless steel and core comprises nitinol.

36. The system of claim 32, wherein the sheath comprises a plurality of filars encircling the core.

37. The system of claim 36, wherein the filars are interlinked to form a hollow braid.

38. A method, comprising:
creating a strengthened region in a wire;
assembling a re-entry device including the wire, the re-entry device having a distal end;
instructing a user of the re-entry device to:
(a) insert the distal end into a lumen defined by an orienting catheter that is extending along the blood vessel,
(b) position the distal end proximate a first aperture, and
(c) rotate the re-entry device until the distal end enters the first aperture;
wherein the strengthened region of the wire is configured such that bending stresses created in the wire during bending about a design bend radius are less than the elastic limit of the wire so that the wire will elastically recover from the bending upon withdrawal from the lumen of the orienting catheter.

39. The method of claim 38 further including instructing a user to insert the distal end of the reentry device into the lumen defined by an orienting catheter, positioning the distal end proximate the first aperture, and rotating the re-entry device until the distal end enters the first aperture.

40. The method of claim 38 further including instructing the user to:
position the distal end of the reentry device proximate a first radiopaque marker of the orienting catheter;
rotate the reentry device until the distal end of the reentry device enters the first aperture of the orienting catheter;
position the distal end of the reentry device between the first radiopaque marker of the orienting catheter and a second radiopaque marker of the orienting catheter;
rotate the reentry device until the distal end of the reentry device enters a second aperture of the orienting catheter; and
advance the distal end through the second aperture of the orienting catheter.

41. The method of claim 38, wherein the re-entry device comprises a proximal leg, a distal leg, and a bend disposed between the proximal leg and a distal leg.

42. The method of claim 39, wherein the bend extends through an angular range that is greater than about 90 degrees and less than about 180 degrees when no external forces are acting on the reentry device.

43. The method of claim 42, wherein the bend extends through an angular range that is greater than about 120 degrees and less than about 150 degrees when no external forces are acting on the reentry device.

44. The method of claim 38, further including instructing the user to direct the distal end of the reentry device towards the blood vessel lumen and into contact with an intimal layer of the blood vessel wall.

45. The method of claim 38, further comprising instructing the user to pierce an intimal layer of the blood vessel wall with the distal end of the reentry device and advancing the distal end of the reentry device into the blood vessel lumen.

46. The method of claim 38, wherein the reentry device comprises a probe extending beyond a distal surface, and the method includes instructing the user to pierce an intimal layer of the blood vessel wall with the probe before the distal surface of the reentry device contacts the intimal layer.

47. The method of claim 38, further comprising instructing the user to withdraw the orienting catheter from the blood vessel wall while a distal portion of the reentry device is extending through an intimal portion of the blood vessel wall.

48. The method of claim 47, further comprising instructing the user to advance a therapy catheter over the reentry device.

49. The method of claim 38, wherein the strengthened region is produced by heat treating the wire.

50. The method of claim 38, wherein the strengthened region is produced by case hardening the wire.

51. The method of claim 38, wherein the strengthened region is produced by cold working the wire.

52. The method of claim 38, wherein the strengthened region is produced by work hardening a portion of the wire.

53. The method of claim 38, wherein the strengthened region is produced by plastically-deforming the wire.

54. The method of claim 38, wherein the strengthened region is produced by twisting the wire.

55. The method of claim 38, wherein the strengthened region is produced burnishing the wire.

56. The method of claim 38, wherein the strengthened region is produced by shot peening the wire.

57. The method of claim 38, wherein the strengthened region is produced by laser shock peening the wire.

58. The method of claim 38, wherein creating a strengthened region in the wire comprises:
providing a laser beam source capable of creating a laser beam;
moving the wire relative to the laser beam source; and
directing a first series of laser pulses to strike an outer surface of a material of the wire.

59. The method of claim 58, wherein each laser pulse striking the outer surface imparts compressive stresses into the material extending below the outer surface.

60. The method of claim 58, wherein the repeated impact of laser pulses on the outer surface of the wire creates the strengthened region, the strengthened region having a generally annular shape so that the strengthened region encircles a central region of the wire.

61. The method of claim 58, wherein:
the first series of laser pulses forms a first series of spots on the outer surface of the wire; and
wherein the spots of the first series are positioned to form a pattern of overlapping spots substantially covering the outer surface of the wire.

62. The method of claim 58, wherein:
the first series of laser pulses forms a first series of spots on the outer surface of the wire; and
wherein the spots of the first series are positioned to form a first helical path along the outer surface of the wire.

63. The method of claim 58, wherein moving the wire relative to the laser beam source comprises translating the wire in a feed direction that is generally parallel to a longitudinal axis of the wire.

64. The method of claim 58, wherein moving the wire relative to the laser beam source comprises simultaneously rotating the wire about a longitudinal axis thereof and translating the wire in a feed direction that is generally parallel to the longitudinal axis.

65. The method of claim 58, further including directing a second series of laser pulses to strike the outer surface of the wire.

66. The method of claim 65, wherein the first series of laser pulses and the second series of laser pulses are both produced by the a single laser beam source.

67. The method of claim 65, wherein the laser beam source is a first laser beam source, and wherein the first series of laser pulses is produced by the first laser beam source, and the second series of laser pulses is produced by a second laser beam source different from the first laser beam source.

68. The method of claim 65, wherein:
the first series of laser pulses forms a first series of spots on the outer surface of the wire, the spots of the first series being positioned to form a first helical path along the outer surface of the wire; and
the second series of laser pulses forms a second series of spots on the outer surface of the wire, the spots of the second series being positioned to form a second helical path along the outer surface of the wire.

69. The method of claim 68, wherein the first generally helical path and the second generally helical path overlap each other to form a pattern of overlapping spots that substantially covers the outer surface of the wire.

70. The method of claim 68, wherein the first generally helical path and the second generally helical path are dimensioned and positioned so as to overlap one another.

71. The method of claim 68, wherein the first generally helical path and the second generally helical path are dimensioned and positioned so that the first series of spots and the second series of spots overlap each other.

72. The method of claim 68, wherein:
the first generally helical path includes a plurality of turns encircling the wire with a first gap between adjacent turns;
the second generally helical path includes a plurality of turns encircling the wire with a second gap between adjacent turns; and
the first gap has a width that is less than a diameter of each spot in the second series and the second gap has a width that is less than the diameter of each spot in the first series so that the first generally helical path and the second generally helical path overlap each other to form a pattern of overlapping spots that substantially covers the outer surface of the wire.
